(19) United States
(12) Patent Application Publication
(10) Pub. No.: US 2013/0314669 A1
(43) Pub. Date: Nov. 28, 2013

(54) METHOD AND SYSTEM FOR ASSESSING VISUAL DISORDER

(71) Applicants: Netta LEVIN, Jerusalem (IL); Noa Raz, Gizo (IL); Tamir Ben-Hur, Jerusalem (IL)

(72) Inventors: Netta LEVIN, Jerusalem (IL); Noa Raz, Gizo (IL); Tamir Ben-Hur, Jerusalem (IL)

(73) Assignee: Hadasit Medical Research Services and Development Ltd., Jerusalem (IL)

(21) Appl. No.: 13/654,557

(22) Filed: Oct. 18, 2012

Related U.S. Application Data
(60) Provisional application No. 61/651,012, filed on May 24, 2012.

(51) Int. Cl. A61B 3/02

(52) U.S. Cl. 351/239; 351/246

(57) ABSTRACT

A method of diagnosis is disclosed. The method comprises using a display device for presenting a motion perception test to a subject and determining a subject response to the motion perception test. The response can be used for assessing presence or absence of demyelination and/or remyelination.

### RELATED APPLICATION

This application claims the benefit of priority under 35 USC 119(e) of U.S. Provisional Patent Application No. 61/651,012 filed May 24, 2012, the contents of which are incorporated herein by reference in their entirety.

### FIELD AND BACKGROUND OF THE INVENTION

The present invention, in some embodiments thereof, relates to medical techniques and, more particularly, but not exclusively, to a method and system for assessing a disorder in the visual system.

The retinal ganglion cell is a retinal output cell, and its-axon is also called an optic nerve fibers, runs in the retinal inner layer and the nerve fibers layer (nearest side to the vitreous body), gathers at the optic disc, leaves the eye ball, forms an optic nerve and undertakes a role of transmitting visual information to the cerebral cortex.

The retinal ganglion cell is distributed over the entire area of the retina. A retinal damage due to, e.g., inflammation and the like can be caused by various disorders. For example, degeneration and damage of the optic nerve can be caused by a disorder such as optic neuritis (ON), capillary angioma of optic disc, ischemic optic neuropathy, defects of retinal nerve fibers layer, retinal optic atrophy, neurotmesis of optic nerve, traumatic optic neuropathy, choked disc, coloboma of optic disc, optic nerve hypoplasia, toxic optic atrophy. A visual disorder can also be caused by atrophy and degeneration of the optic nerve, e.g., as a result of elevated intraocular pressure.

Optic neuritis is defined as inflammation of the optic nerve. It is one of the causes of acute loss of vision associated with pain. The diagnosis of ON is usually made clinically. The classic clinical presentation of ON consists of (a) loss of vision, (b) eye pain, and (c) dyschromatopsia, which refers to the impairment of accurate color vision. Seventy percent of cases in adults are unilateral.

Optic neuritis is caused by demyelination and can be idiopathic and isolated. This condition is considered transient when using standard visual testing. However, subjects typically continue to perceive difficulties in performing everyday visual tasks following an ON attack. Optic neuritis has an association with multiple sclerosis (MS), wherein about 20% of cases of MS manifest as ON, and 38-50% of patients with MS develop ON at some point during the course of their disease.

ON diagnosis is typically based on clinical presentation, but finds manifestation also in other modalities such as diagnostic imaging, and electrophysiology.

Diagnostic imaging using magnetic resonance imaging (MRI) is a sensitive technique, possibly revealing areas of the brain that have lost myelin. An MRI scan may even distinguish areas of active, recent demyelination from areas in which demyelination took place some time ago. Recently, functional MRI (fMRI) has been used to demonstrate dynamic relationships among structure, clinical outcome, and functional activation. fMRI was used to evaluate the cortical response following an ON attack [Werring et al., Journal of neurology, neurosurgery, and psychiatry 2000, 68(4):441-449; Toosy et al., Annals of neurology 2005, 57(5):622-633; Levin et al., Neuroimage 2006, 33(4):1161-1168; Korsholm et al., Brain 2007, 130(Pt 5):1244-1253].

Electrophysiology includes the measurement of the electrical activity of neurons, particularly action potential activity. In an evoked potentials (EP) test, electrical responses in the brain are recorded when nerves are stimulated. For example, visual evoked potentials (VEP) are the brain's electrical response to a visual stimulus. Normally, the brain responds to a stimulus with characteristic patterns of electrical activity. In subjects with ON, the response may be slower because signal conduction along demyelinated nerve fibers is impaired. VEP amplitudes, which are believed to reflect the number of functional optic nerve fibers, are typically reduced in the acute phase of ON, but recover within 3 months along with the recovery of visual acuity. VEP amplitudes were found to correlate with standard visual measures, such as visual acuity, color vision, visual field and contrast sensitivity. Correlations were also found with retinal nerve fiber layer (RNFL) thickness, as measured by optical coherence tomography (OCT). VEP latency prolongation typically characterizes the chronic stages of ON, even after standard visual functions have returned to normal.

### SUMMARY OF THE INVENTION

According to an aspect of some embodiments of the present invention there is provided a method of diagnosis. The method comprises using a display device for presenting a motion perception test to a subject, determining a subject response to the motion perception test, and correlating the response to visual evoked potentials latency.

According to some embodiments of the invention the method further comprises assessing presence or absence of demyelination based on the response. According to some embodiments of the invention the method further comprises assessing presence or absence of remyelination based on the response.

According to an aspect of some embodiments of the present invention there is provided a method of diagnosis. The method comprises using a display device for presenting a motion perception test to a subject, determining a subject response to the motion perception test, and assessing presence or absence of demyelination and/or remyelination based on the response.

According to some embodiments of the invention the determining the subject response comprises applying a scoring procedure to assign a score to the response.

According to some embodiments of the invention the method comprises assessing prolongation of the visual evoked potentials latency.

According to some embodiments of the invention the method is executed while the subject is in an acute phase of optic neuritis.

According to some embodiments of the invention the subject has an optic neuritis history, and the method is executed at least one month following an acute phase of the optic neuritis.

According to some embodiments of the invention the subject has an optic neuritis history, wherein the method is executed less than two months following an acute phase of the optic neuritis, and wherein the method comprises predicting visual recovery of the subject at a future time.

According to some embodiments of the invention the prediction is based on a predetermined recovery rate.

According to some embodiments of the invention the motion perception test comprises a motion detection test.

According to some embodiments of the invention the motion detection test comprises displaying a stimulus selected from the group consisting of a coherent moving dot array and a collection of stationary dots.

According to some embodiments of the invention the motion detection test comprises displaying a plurality of stimuli, each stimulus consisting of a coherent moving dot array characterized by a different moving velocity.

According to some embodiments of the invention the motion perception test comprises an object from motion (OFM) extraction test.

According to some embodiments of the invention the OFM extraction test comprises displaying at least one OFM stimulus consisting of an array of dots outlining a patterned object and being at a relative motion relative to a patterned background, the patterned object and the patterned background being characterized by the same pattern and being indistinguishable in the absence of the relative motion.

According to some embodiments of the invention the at least one OFM stimulus comprises a plurality of OFM stimuli, each being characterized by a different motion velocity.

According to an aspect of some embodiments of the present invention there is provided a method of assessing the effect of a treatment. The method comprises administering to a subject a drug identified for the treatment of demyelinating condition; presenting a motion perception test to the subject; determining a subject response to the motion perception test; assessing presence, absence or level of at least one of (i) demyelination and (ii) remyelination, based on the response, thereby providing an assessment; and assessing the effect of the drug based, at least in part, on the assessment.

According to an aspect of some embodiments of the present invention there is provided a system for diagnosis. The system comprises a display device and a data processor configured for displaying a motion perception test, receiving a subject response to the motion perception test, correlating the response to visual evoked potentials latency, and generating output pertaining to the correlation.

According to an aspect of some embodiments of the present invention there is provided a computer software product. The computer software product comprises a computer-readable medium in which program instructions are stored, which instructions, when read by a computer, cause the computer to display a motion perception test, to receive a subject response to the motion perception test, to correlate the response to visual evoked potentials latency, and to generate output pertaining to the correlation.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart diagram describing a method suitable for diagnosis according to some embodiments of the present invention;

FIGS. 2A-B are schematic illustrations describing an object-from-motion (OFM) extraction test, according to some embodiments of the present invention;

FIG. 3 is a diagram illustrating a time line used in experiments performed according to some embodiments of the present invention;

FIGS. 4A-D show sustained deficit in motion perception as obtained during experiments performed according to some embodiments of the present invention;

FIG. 5 shows fMRI activation maps which describe activation obtained during experiments performed according to some embodiments of the present invention 12 months following the acute phase of ON;

FIG. 6 shows fMRI activation maps which describe activation as obtained in experiments performed according to some embodiments of the present invention during the acute phase of ON, as obtained during experiments performed according to some embodiments of the present invention;

FIGS. 7A-B are differential fMRI activation maps showing cortical activation for controls versus ON patients, obtained in experiments performed according to some embodiments of the present invention during static object and OFM viewing;

FIGS. 8A-B show fMRI activation levels obtained in experiments performed according to some embodiments of the present invention during the acute (FIG. 8A) and 12 months (FIG. 8B) phases of ON;

FIGS. 9A-D show performance levels in different OFM speeds (referred to as dot's velocity in FIGS. 9A-D), obtained during experiments performed according to some embodiments of the present invention in the acute (FIG. 9A), 1 month (FIG. 9B), 4 months (FIG. 9C) and 12 months (FIG. 9D) phases of ON;

FIGS. 10A-E show VEP measurements, static and dynamic visual functions throughout a 12 month follow-up experiment performed according to some embodiments of the present invention;

FIGS. 11A-B show additional visual field and color perception obtained throughout a 12 month follow-up experiment performed according to some embodiments of the present invention;

FIGS. 12A-F show performance levels in visual acuity (FIGS. 12A and 12D), contrast sensitivity (FIGS. 12B and 12E) and OFM tasks (FIGS. 12C and 12F) obtained in experiments performed according to some embodiments at the 1 month time point, plotted against performance level assessed at the 4 month phase (FIGS. 12A-C) and 12 month phase (FIGS. 12D-F);

FIGS. 13A-C show visual measurements as a function of VEP amplitudes and latencies obtained in experiments performed according to some embodiments; and

FIGS. 14A-D show correlation between changes in VEP measurements and visual functions as obtained in experiments performed according to some embodiments.

### DESCRIPTION OF SPECIFIC EMBODIMENTS OF THE INVENTION

The present invention, in some embodiments thereof, relates to medical techniques and, more particularly, but not exclusively, to a method and system for assessing a disorder in the visual system.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In experiments conducted by the present Inventors, it was uncovered that for subjects who had experienced a visual disorder, particularly ON, VEP latency tend to remain significantly prolonged and motion perception tend to remain impaired for a long period of time following the acute phase of the visual disorder. The present Inventors also found that for a partially recoverable visual disorder, such as ON, the prolonged VEP latency and impaired motion perception are observed even after VEP amplitudes and static visual functions have been recovered.

The present inventors found that VEP latencies correlate with motion perception. VEP latencies generally reflect nerve conduction levels and the present Inventors, without wishing to be bound to any particular theory, postulated that the surprisingly observed correlation between motion perception and VEP latencies can be used, in some embodiments of the present invention, for assessing presence or absence of demyelination and/or remyelination.

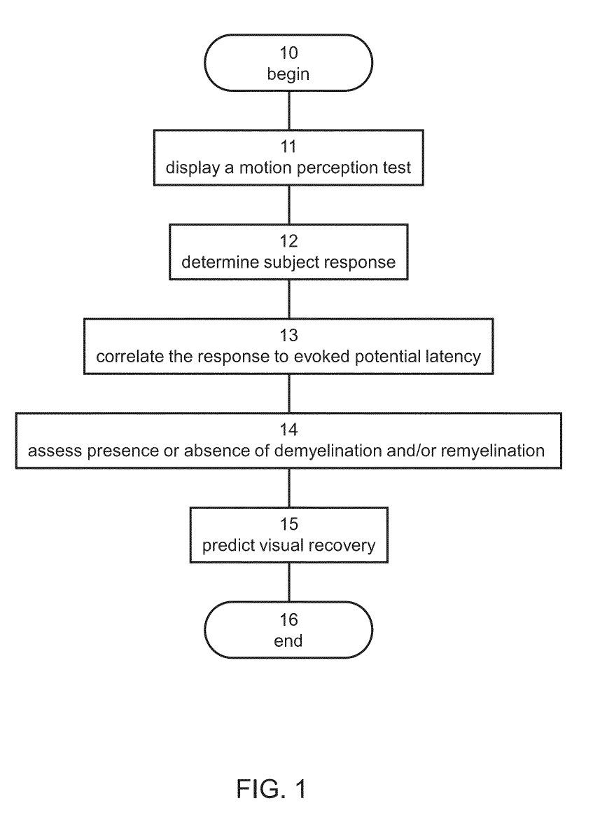

Referring now to the drawings, FIG. 1 is a flowchart diagram illustrating a method of diagnosis, according to some embodiments of the present invention.

It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method begins at 10 and continues to 11 at which a motion perception test is presented to a subject. The motion perception test can be presented using a display device. For example, a data processor, such as a general purpose computer or dedicated circuitry, can be configured for transmitting to the display device imagery data pertaining to the motion perception test.

The present embodiments contemplate more than one type of a motion perception test.

In some embodiments of the present invention, a motion detection test is presented to the subject. For example, the method can display two types of stimuli, in a non-simultaneous manner, wherein a first type of stimulus is a coherent moving dot array, and a second type of stimulus is a collection of stationary dots. The subject can then be asked to indicate whether he or she detects a motion in the displayed stimulus. Typically, but not necessarily, the coherent moving dot array forms a moving noise over a stationary background.

In some embodiments of the present invention the method displays a series of stimuli. Some of the stimuli in the series can be of the first type and some can be of the second type. In some embodiments, the motion velocity or speed can be varied over the series.

In experiments performed by the present inventors six different speeds, ranging from slow speed of about 0.05 degrees/s to high speed of about 2 deg/sec were employed. The direction of motion can optionally and preferably be varied between two or more successive stimuli. For example, half of the stimuli can be displayed as moving leftward and half can be displayed as moving rightward.

In some embodiments of the present invention, an object-from-motion (OFM) extraction test is presented to the subject. Broadly speaking, an OFM test is a displayed image showing an object over a background wherein there is a relative motion between the object and the background. Optionally and preferably the object and background are selected such that they are indistinguishable in the absence of the relative motion.

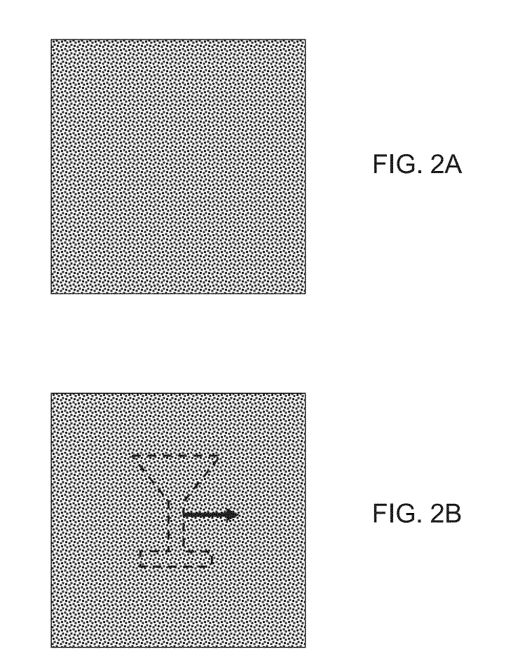

For example, the method can display an OFM stimulus consisting of an array of dots outlining a patterned object and being at a relative motion relative to a patterned background, where the patterned object and the patterned background are characterized by the same pattern (e.g., a dotted pattern) such that they are indistinguishable in the absence of the relative motion. A representative example is illustrated in FIGS. 2A-B. FIG. 2B shows a patterned object over a patterned background. Both the object and the background are patterned in identical manner (dots, in the present Example). In FIG. 2B, the object is shown outlined by a dashed line, and its motion direction is illustrated by a right pointing arrow. However, during the OFM in the present embodiment, no outline and no arrow are presented to the subject, so that any identification of the moving object is solely by virtue of its motion. When the object is static relative to the background, the object cannot be distinguished from the background, as illustrated in FIG. 2A. Thus, the object recognition is dependent on motion perception of the subject.

The shape of the object is preferably familiar to the subject, and can be selected from the group consisting of geometrical shapes, two-dimensional drawings, numbers, letters and the like. The subject can be asked to identify the object and name it.

In some embodiments of the present invention a series of OFM stimuli is presented to the subject. In some embodiments, the motion velocities or speeds of the OFM stimuli can be varied over the series. In experiments performed by the present inventors six different speeds, ranging from slow speed of about 0.05 degrees/s to high speed of about 2 deg/sec were employed. The direction of motion can optionally and preferably be varied between two or more successive stimuli. For example, half of the stimuli can be displayed as moving leftward and half can be displayed as moving rightward.

In various exemplary embodiments of the invention the subject is presented with a motion detection test including a plurality of motion detection stimuli, as well as with an OFM extraction test including a plurality of OFM stimuli. The number of motion detection stimuli presented to the subject in a single motion detection test is typically from about 20 stimuli to about 60 stimuli. The number of OFM stimuli presented to the subject in a single OFM extraction test is typically from about 20 OFM stimuli to about 60 OFM stimuli.

The method continues to 12 at which a subject response to the motion perception test is determined. For example, following each stimulus presentation, a record regarding the detection and/or identification or lack thereof can be made. This can be performed automatically using dedicated software, configured to receive input from the subject or an operator over a predetermined time-window following the presentation of the stimulus.

In some embodiments of the present invention a scoring procedure is applied so as to assign a score to the response of the subject. For example, each stimulus can be assigned with a stimulus weight, and the sum of weights of all identified or detected stimuli can be set as the subject response score. Unidentified stimuli can also be assigned with a weight (e.g., zero or negative weight). Typically, the task difficulty in the motion perception test is dependent on the stimulus velocity or speed, wherein the difficulty decreases as the speed increases. Thus, according to some embodiments of the present invention, the weight of a particular stimulus is set based on the speed of the stimulus, with higher weights to lower speeds.

Following is a representative motion perception test protocol which can be employed according to some embodiments of the present invention. The subject is first presented with a plurality of stimuli, all shown at the lowest speed. Stimuli which are identified at this speed are marked and are not presented again during the test. Those stimuli which are not identified are then presented at the next to lowest velocity and so on, until they are identified or until the highest speed is reached.

As a representative example for the scoring procedure, consider a test in which N different speeds v₁, v₂, . . . , v_{N} are employed, where v₁ is the lowest speed, v₂ is the next to lowest speed and so on. Let w₁, w₂, . . . , w_{N}, be the respective weights associated with the speeds, where w₁>w₂> . . . >w_{N} (for example, the weight wᵢ of the ith speed vᵢ can be set to N−i+1, where i=1, . . . , N), and let wₒ be the weight associated with an unidentified or undetected stimulus. Consider further that K stimuli are presented to the subject at the speed v₁. Suppose that k₁ (0≦k₁≦K) stimuli are identified at speed v₁, so that these stimuli are not presented again. At the speed v₂, only the remaining K-k₁ stimuli are presented. Suppose further that k₂ (0≦k₂≦K) stimuli are identified at speed v₂, so that at speed v₃, only the remaining K-k₁-k₂ stimuli are presented. The procedure optionally and preferably continues until all the stimuli are identified or until the remaining stimuli are presented to the subject at speed v_{N}. The response score can then be set to w₁k₁+w₂k₂+ . . . +w_{N}k_{N}+w₀k₀, where k₀ is the number of unidentified or undetected stimuli and k₀+k₁+k₂+ . . . +k_{N}=K. The response score can therefore be from zero (no stimulus identified) to w₁K (all stimuli identified at the lowest speed, hence received the highest score).

As a numerical example, suppose that in an OFM extraction test, N=6, wᵢ=7-i and w₀=0, and 20 OFM stimuli are presented at the lowest speed vᵢ, so that K=20. Suppose further that no stimulus is identified at speeds vᵢ and v₂, 1 stimulus is identified at each of speeds v₃ and v₄, 7 stimuli are identified at speed v₅, 9 stimuli are identified at speed v₆, and 2 stimuli are not identified at all. The response score in this numerical example is, therefore, 0*6+0*5+1*4+1*3+7*2+9*1+2*0=30, where the term at the right hand side corresponds to the two unidentified stimuli.

Also contemplated are embodiments in which the score is based on the percentage or ratio of the correctly identified stimuli relative to the total number of stimuli. The percentage or ratio is optionally and preferably calculated separately for each speed and each type of test. Once all the percentages or ratios are calculated, they can be combined to provide a score. For example, the score can be a sum or a weighted sum of the percentages or ratios. When a weighted sum is employed, the weights are optionally and preferably higher for lower speeds, as further detailed hereinabove.

Alternatively, the percentage or ratio can be calculated globally for all the stimuli. For example, the core can equal the number of correctly identified stimuli divided by the total number of stimuli that are presented to the subject.

The method optionally and preferably continues to 13 at which the response is correlated to VEP latency. When a response score is calculated, the score is preferably correlated to the VEP latency. Typically, higher response score is correlated to shorter VEP latency. The correlation result can be output to a display device or transmitted to a computer readable medium. The correlation result can be quantitative or qualitative. A qualitative result can include an indication that the VEP latency is higher, lower or within the normal range of VEP latencies for the particular subject under diagnosis. If a history of VEP latencies for the particular subject under diagnosis is accessible, the correlation result can be expressed relative to this history. A representative example of a quantitative correlation result includes an assessment of the prolongation of the VEP latency.

In some optional embodiments of the present invention, the method, at 14, assess presence or absence of demyelination and/or remyelination based on the response. For example, when the subject has difficulty in the identification and/or detection of slow motion, the method can determine that it is likely that the subject is under demyelination process, and when the subject successfully identifies both slow motion and fast motion, the method can determine that it is likely that the subject is under remyelination process.

The present embodiments are particularly useful for the assessment of subject suffering from ON. Thus, the method can be executed while the subject is, or being suspected as being, in an acute phase of ON, so as to assess whether or not a demyelination process is present.

The method can also be executed after the acute phase of ON, for example, at least one month or at least two months or at least three months or at least four months or at least five months or at least six months or at least twelve months after the acute phase of ON. In these embodiments, the method can assess whether or not the dynamic visual function of the subject has been recovered. The present Inventors found that for subjects with ON, dynamic visual function remain impaired even when conventional techniques directed to the assessment of static visual function indicate that the visual acuity of the subject has been recovered. Without wishing to be bound to any particular theory, it is believed that sustained motion perception deficit following ON explains the continued visual complaints of patients long after recovery of visual acuity.

While reducing the present invention to practice it has been unexpectedly uncovered that following a 1-month time point after the acute phase of ON, the rate of improvement in the response score as calculated from the subject response to of the motion perception, was similar across many ON patients. The rate of recovery can be considered universal and be used for predicting the visual recovery of the subject.

Thus, according to some embodiments of the present invention the method continues to 15 at which the method predicts the visual recovery of the subject at a future time based on the response as determined less than two months (for example, about 1-2 months) after the acute phase of ON. This can be done by extrapolation, using the universal recovery rate. For example, denoting the response score at 1-month after the acute phase by S(1) and the universal recovery rate by R, the response score for a future time t, where t is more than 1 month more preferably more than 2 months, is S(1)+Rt. A typical value for R is from 0.05 per three months to about 0.2 per three months, e.g., 0.12 per three months.

The method ends at 16.

While the embodiments above are described with a particular emphasis to ON, it is to be understood that more detailed reference to ON is not to be interpreted as limiting the scope of the invention in any way. Thus, selected operations of the method are also suitable for other visual disorders, including, without limitation, a visual disorder or a disease with various symptoms of loss of vision, low vision, narrow vision, abnormal color sensation and misty vision, abnormal electroretinogram, and visually evoked potential and the like, which is caused by decreased optic nerve fibers due to damage, degeneration, and the like; a visual disorder accompanying degeneration or damage of optic nerve (optic neuritis, capillary angioma of optic disc, ischemic optic neuropathy, defects of retinal nerve fibers layer, retinal optic atrophy, neurotmesis of optic nerve, traumatic optic neuropathy, choked disc, coloboma of optic disc, optic nerve hypoplasia, toxic optic atrophy, damage due to pseudo tumor cerebri etc.); visual disorder due to optic atrophy, degeneration and the like caused by elevated intraocular pressure (glaucoma etc.) and the like.

According to some embodiments of the present embodiments there is provided a method suitable for assessing the effect of a treatment. The method comprises administering to a subject a drug identified for the treatment of demyelinating condition. The drug can comprise, for example, an immunomodulatory compound. Such compound can either be commercially purchased or prepared according to the methods known in the art. Suitable compounds include immunomodulatory compounds that are racemic, stereomerically enriched or stereomerically pure, and pharmaceutically acceptable salts, solvates, stereoisomers, and prodrugs thereof. Suitable compounds may be small organic molecules having a molecular weight less than about 1,000 g/mol.

The drug can include an antidemyelination agent including beta-interferon (such as AVONEX®. which is available from Biogen, Inc. and BETASERON® which is available from Berlex Laboratories), which can decrease the frequency and occurrence of flare-ups and slow the progression to disability, glatiramer acetate (such as COPAXONE® which is available from Teva Neuroscience, Inc.), which can reduce the frequency of relapses, and/or administration of corticosteroids, such as prednisone (available from Roxane), to relieve acute symptoms. The amount of respective antidemyelination agent to be administered to the subject readily can be determined by one skilled in the art from the Physician's Desk Reference (56^{th} Ed. 2002) at pages 1013-1016,988-995, 3306-3310 and 3064-3066, incorporated herein by reference.

The drug can alternatively or additionally include at least one compound selected from the group consisting of natalizumab (Tysabri®), fingolimod (Gilenya®), laquinimod, cladribine and dimethylfumarate.

Natalizumab is a humanized monoclonal antibody against alpha-4 integrin, which is required for white blood cells to move into organs. Natalizumab's mechanism of action is believed to be the inhibition of immune cells from crossing blood vessel walls to reach affected organs. Natalizumab has proven effective in treating the symptoms of both multiple sclerosis and Crohn's disease, preventing relapse, vision loss, cognitive decline and improving quality of life in people with multiple sclerosis. It increases rates of remission and preventing relapse in Crohn's disease. Natalizumab is typically administered by intravenous infusion every 4 weeks.

Fingolimod is an FDA approved S1P1 modulator for the treatment of multiple sclerosis and has shown beneficial effects in several preclinical models (e.g., cerebral ischemia, cancer, organ transplantation. Fingolimod becomes active in vivo following phosphorylation by sphingosine kinase 2 to form Fingolimod-P phosphate, which resembles the ligand S1P and competes with it to bind to four of the five S1P receptors.

Laquinimod, a 1, 2-dihydroquinoline derivative, is a once-daily, orally administered immunomodulatory compound that is being developed as a disease-modifying treatment for MS.

Cladribine (2-chlorodeoxyadenosine), a purine analog, is a synthetic anti-cancer agent that also suppresses the immune system. An oral pill form has been successfully tested for multiple sclerosis.

Dimethylfumarate (BG-12), an α,β-unsaturated ester, reacts rapidly with the detoxifying agent glutathione by Michael addition. It is reported to have potential neuroprotective and anti-inflammatory effects according to a phase IIb clinical trial for the treatment of relapsing-remitting multiple sclerosis. In clinical trials doses up to 240 mg tds of BG-12 have been effective in relapsing-remitting multiple sclerosis.

It is expected that during the life of a patent maturing from this application many relevant medicaments will be developed and the scope of the term “drug identified for the treatment of demyelinating condition” is intended to include all such new technologies a priori.

Once the drug is administered to the subject, selected operations of the method described above with respect to the flowchart diagram shown in FIG. 1 can be executed, so as to provide an assessment regarding the presence, absence or level of demyelination and/or remyelination. The effect of drug can then be assessed, based, at least in part, on the provided assessment. For example, when the provided assessment indicates the presence of remyelination, the method can output a report that the drug is likely to be effective, and when the provided assessment indicates the presence of demyelination, the method can output a report that the drug is likely to be ineffective.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

The present Example describes experiments performed according to some embodiments of the present invention to assess the recovery process in patients after an acute ON attack, and to compare static and dynamic visual functions.

Motion perception begins in the retina, mediated through the magnocellular pathway, containing cells with transient responses and fast-conductive axons. Cortically, the visual area MT (middle temporal), likely plays a major role in the integration of local motion signals into global percepts [7].

Recently, fMRI was used to evaluate the cortical response following an ON attack [8-12], suggesting that changes in cortical organization may have an adaptive role in visual recovery after ON, in addition to the remyelinating process in the nerve itself.

The present study assesses motion perception longitudinally following an ON attack, and to document its associated cortical response.

### Methods

The Hadassah Hebrew University Medical Center Ethics Committee approved the experimental procedure. Written informed consent was obtained from all subjects.

The study was carried out at the Hadassah Hebrew University Medical Center, Jerusalem, Israel. Twenty-one patients aged 18-41 (mean±STDEV 28.9±6.6) years presenting with a first-ever episode of acute ON were enrolled.

The whole follow-up process was carried out at the Hadassah medical center. This was done at fixed intervals: 1, 4 and 12 months following the acute phase. Out of the patients' group, all but one who had a recurrent attack, succeed to the 1 month phase. Fifteen and thirteen patients succeeded to the 4 and 12 months phases, respectively. As whole, 2 patients were excluded from the study due to a recurrent attack and the rest were not willing to precede the follow-up process. Twenty-one control subjects who matched the patients for age, gender and dominant eye on a subject-by-subject basis were included in the study. Control patients were tested in the behavioral and the fMRI study.

Table 1, below summarizes the patient characteristics. In Table 1, visual acuity represent values measured at presentation; MS is abbreviation for Multiple Sclerosis; CIS is abbreviation for Optic Neuritis as a clinically isolated syndrome; DIS corresponds to MRI examination is space and DIT corresponds to MRI examination is time according to the McDonald criteria [Polman C H, Reingold S C, Edan G, et al. Diagnostic criteria for multiple sclerosis: 2005 revisions to the “McDonald Criteria”. Annals of neurology 2005; 58:840-846]; and asterisks define patients who participated in the fMRI study in addition to the behavioral follow-up. All patients who had participated in the behavioral follow-up and gave their informed consent were enrolled to the fMRI study described below.

TABLE 1

| | | | Visual | | | | | Recurrent |
|---|---|---|---|---|---|---|---|---|
| ID | age | gender | acuity | Disc | RAPD | Diagnosis | MRI | attack |
| 1 | 34 | M | 1.2 (6/5) | | + | CIS | DIS | |
| 2 | 33 | M | 0.1 (6/60) | | + | CIS | — | |
| 3 | 32 | F | FC 0.5 m | swelling | + | CIS | — | |
| 4* | 33 | F | 1 (6/6) | | + | Behcet's | | Fellow eye |
| | | | | | | syndrome | | (1 month) |
| | | | | | | (2003) | | |
| 5* | 31 | F | FC 0.5 m | swelling | + | CIS | DIS | |
| | | | | | | | DIT | |
| 6* | 26 | M | FC 0.25 m | swelling | + | CIS | — | |
| 7* | 32 | F | 0.2 (6/30) | | + | CIS | — | |
| 8* | 20 | F | 1.2 (6/5) | swelling | + | CIS | — | |
| 9* | 33 | M | FC 0.5 m | | + | CIS | DIS | |
| | | | | | | | DIT | |
| 10* | 29 | M | 0.1 (6/60) | | trace | CIS | — | Same eye |
| | | | | | | | | (4 months) |
| 11* | 27 | F | 0.2 (6/30) | | + | CIS | — | |
| 12* | 41 | M | FC 0.25 m | | + | CIS | — | |
| 13* | 18 | M | 0.8 (6/6) | | + | CIS | DIS | |
| | | | | | | | DIT | |
| 14* | 29 | F | 1 (6/6) | | + | MS | | |
| | | | | | | (2004) | | |
| 15* | 22 | F | FC 0.5 m | swelling | + | CIS | DIS | |
| 16 | 20 | F | 0.6 (6/10) | | trace | CIS | DIS | |
| | | | | | | | DIT | |
| 17 | 21 | M | 0.8 (6/7.5) | swelling | + | CIS | — | |
| 18* | 27 | M | FC 0.3 m | | + | CIS | DIS | |
| 19 | 41 | F | 0.6 (6/10) | | trace | CIS | — | |
| 20 | 30 | F | 0.8 (6/7.5) | | + | CIS | — | |
| 21 | 32 | F | 0.8 (6/7.5) | | + | CIS | — | |

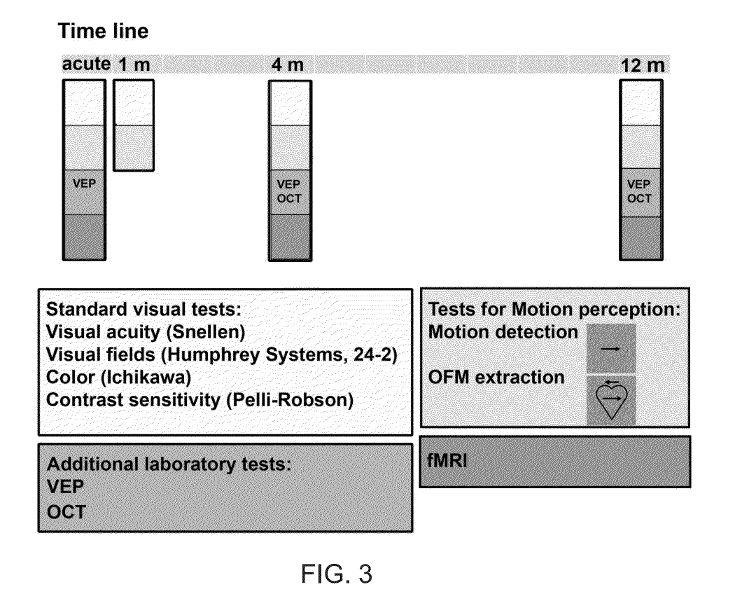

Four types of examination were performed, as described below. Subjects were evaluated monocularly in each test, according to the timeline described in FIG. 3.

### (a) Standard Visual Tests

Standard visual tests included Visual Acuity (VA, measured by Snellen visual-acuity chart); Visual Fields Estimation (by the automatic Humphrey's perimetry visual-field test 24-2), Color Perception (Standard pseudoisochromatic plates, by Ichikawa) and Contrast Sensitivity (CS, Pelli-Robson chart at 1 meter, Metropia Ltd., Cambridge, UK).

### (b) Additional Tests

Additional included (i) Optical Coherence Tomography (OCT), wherein the retinal nerve fiber layer (RNFL) thickness was recorded on a Zeiss Stratus OCT 3 with version 4 software; and (ii) Pattern Visual Evoked Potentials (VEPs), wherein the amplitudes and latencies of the major positive component (P100) were recorded to pattern reversal full-field checkerboards.

Patients in whom the VEP waveform was unobtainable due to poor vision were excluded from the VEP latency analyses (n=7 in the acute phase and n=2 in later phases). Additionally, due to the wide range of variability within a normal population, to best study the effect of ON over time, VEP amplitudes from the affected eye (AE) were expressed as a percentage of that from the fellow eye (FE).

Patients' performance level in the standard visual and additional laboratory tests was compared to the mean normal population values, when available from the literature (in the VA, CS, VEP and OCT measures). For the visual fields and color perception measures, patients' performance levels were compared to the optimal score available in each test (see Table 2 and Table 3 for details). Note that comparison with the mean normal population value or the optimal score is a rigorous criterion. In the clinical constellation, normal visual levels are defined as those above the lower limit of the normal range. A delta score, representing the differences between the subject's data and the given norm, was calculated for each subject. This was done separately for the affected and fellow eyes. Significant differences were defined when the deltas of the group were significantly different from zero.

### (c) Motion Perception Tests

These tests included Motion detection and OFM extraction. A random pattern of dots of near 100% contrast was generated on computer.

An array of dots, sized 15*14 cm, was moved at a homogeneous velocity (out of six possible velocities). The dots were switched on the instant that they started moving and were switched off immediately following presentation

In the motion detection test the subjects were presented with either coherent moving dot arrays (moving noise) or stationary dots and were asked to state whether or not they identified movement in each stimulus. Coherent moving noise was presented at six different speeds: 0.05, 0.1, 0.25, 0.5, 1 and 2 degrees/s. Half of the stimuli were moving leftward and half moving rightward. However, only the lower 3 speeds were included in the data analysis, being the most sensitive measure.

The OFM extraction test is a variation of the one used in Ref [5]. Subjects viewed motion-defined objects and were asked to recognize and name the object. An array of dots composed an object, by moving the dots within the image rightward while moving the dots outside the image leftward at V degrees/s or vice versa. The motion-defined objects were composed from a random pattern of dots of near 100% contrast.

The exact pattern of dots was used for the image and background, resulting in a camouflaged object that cannot be detected when the dots are stationary or moving as a whole. Thus, object recognition is dependent on motion perception.

While only the OFM stimuli were included in the data analysis presented below, two additional conditions were presented to the subjects during this test. A first additional condition included coherent moving noise stimuli (the same array of dots moving as a whole, so that motion but no object is apparent). These were presented as “foil trails”. A second additional condition included static objects, in which objects' contours are defined by luminance difference. These were presented in order to determine subjects' naming skills, and rule out a naming bias which may interfere with the results of the OFM condition.

OFM and coherent moving noise stimuli were moved at six different speeds: 0.05, 0.1, 0.25, 0.5, 1 and 2 degrees/s. The difficulty of the task decreased as velocities increased. Half of the stimuli were moving leftward and half moving rightward. However, only the lower 3 velocities were included in the data analysis, as for the motion detection task. Each experimental block included 60 OFM stimuli (20 at each velocity), 12 moving noise stimuli (4 at each velocity) and 10 static objects. To avoid between-eye and between-phase learning, 4 experimental blocks were created, each consisting of different stimuli. Thus, the two eyes of a subject were shown different blocks on each run, and each eye was shown different blocks on adjacent runs. The exact experimental block (1-4) performed by a patient was also done by his control subject, matched on the basis of the tested eye. That is, the block shown to the dominant eye of a patient was also presented to the dominant eye of his control subject (and the same for the non-dominant eye). This was done in each testing phase.

In the motion detection and OFM extraction tests, stimuli were presented on a computer screen situated at a distance of about 50 cm from subjects' eyes. Stimuli were presented in a random order, each preceded by a 980 ms long fixation and lasting until the subject responded or for a maximum of 4 seconds.

The percentile of correct responses was calculated for each subject and then averaged across subjects. A delta score, representing the difference between the patient and his matched control, was calculated. Significant differences were defined as cases in which the deltas of the group were significantly different from zero.

To address the relative deficit of the AEs in the different visual measures, the performance level was further represented at all visual measures in a percent correct scale (actual performance/optimal score available in the test, see Table 2 and Table 3).

A repeated measures analysis of variance (ANOVA) with a within groups factors of eye (AE vs. FE), test (VA, CS, color perception, visual field, OFM and motion detection) and time since the event (0 or 4 months) was used to compare changes along time in the different visual measures. This was performed using SPSS 11.0 for Windows (SPSS, Chicago, Ill., USA).

Tables 2 and 3 below summarize the visual tests performed in the affected eye (Table 2) and the fellow eye (Table 3).

TABLE 2

| | median (range) | | | | normal |
|---|---|---|---|---|---|
| | Acute | 1 month | 4 months | 12 months | values |
| n | 21 | 21 | 15 | 13 | |
| Visual | 0.4 | 1 | 1 | 1.2 | 1 |
| acuity | (0.0025-1.2) | (0.005-1.5) | (0.005-1.5) | (0.005-1.5) | |
| (Snellen) *^{a} | 40 | 100 | 100 | 100 | 100% |
| | (0.25-100%) | (0.5-100%) | (0.5-100%) | (0.5-100%) | |
| | p = 6*10⁻⁵ | | | | |
| visual field | 94 | 100 | 100 | 100 | 100% |
| (0-10°) *^{b} | (0-100%) | (13-100%) | (13-100%) | (38-100%) | |
| (Humphrey) | p = 0.003 | | | | |
| Color | 47.5 | 100 | 100 | 100 | 100% |
| (Ichikawa) *^{c} | (0-100%) | (0-100%) | (0-100%) | (0-100%) | |
| | p = 0.001 [4] | | | | |
| Contrast | 1.35 | 1.65 | 1.65 | 1.8 | 1.84 *^{f} |
| sensitivity | (0-1.95) | (0-1.95) | (0-1.95) | (0-1.95) | |
| (Pelli- | 69.2 | 84.6 | 84.6 | 92.3 | 94.4% |
| Robson) *^{d} | (0-100%) | (0-100%) | (0-100%) | (0-100%) | |
| | p = 7*10⁻⁵ | p = 0.01 | | | |
| MD *^{e} | 23.6 | 55.5 | 72.2 | 55.6 | 84% |
| | (0-83.3%) | (0-100%) | (0-100%) | (0-100%) | |
| | p = 5*10⁻⁷ [3] | p = 0.0007 [3] | p = 0.047 [3] | p = 0.02 [1] | |
| OFM *^{e} | 5 | 26.7 | 33.3 | 33.3 | 59.5% |
| | (0-46.5%) | (0-60%) | (0-66.7%) | (0-68.3%) | |
| | p = 4*10⁻⁸ | p = 9*10⁻⁶ | p = 0.0008 [1] | p = 0.002 | |
| OCT (μm) | | | 97 | 75.25 | 100.1*^{g} |
| | | | (51.7-107.3) | (36-102.8) | |
| | | | [3] | p = 0.004[1] | |
| VEP | 67.2 | | 94.8 | 116.5 | |
| amplitude: | (0-137.3%) | | (56.6-192.9%) | (57.2-172%) | |
| AE/FE | p = 0.001 | | | | |
| VEP | 145 | | 138 | 137 | 103.8 *^{h} |
| latency(ms) | (133-166) | | (122-151) | (126-140) | |
| | p = 7*10⁻⁷ | | p = 2*10⁻⁷ | p = 3*10⁻⁵ | |

TABLE 3

| | median (range) | | | | normal |
|---|---|---|---|---|---|
| | Acute | 1 month | 4 months | 12 months | values |
| n | 21 | 21 | 15 | 13 | |
| Visual | 1 | 1.2 | 1.2 | 1.2 | 1 |
| acuity | (0.8-1.5) | (0.8-1.5) | (1-1.5) | (1-1.5) | |
| (Snellen) *^{a} | 100 | 100 | 100 | 100 | 100% |
| | (80-100%) | (80-100%) | (100-100%) | (100-100%) | |
| visual field | 100 | 100 | 100 | 100 | 100% |
| (0-10°) *^{b} | (100-100%) | (100-100%) | (100-100%) | (100-100%) | |
| (Humphrey) | | | | | |
| Color | 100 | 100 | 100 | 100 | 100% |
| (Ichikawa) *^{c} | (100-100%) | (100-100%) | (100-100%) | (100-100%) | |
| | [4] | | | | |
| Contrast | 1.85 | 1.95 | 1.95 | 1.95 | 1.84 *^{f} |
| sensitivity | (1.8-1.95) | (1.65-1.95) | (1.65-1.95) | (1.8-1.95) | |
| (Pelli- | 94.9 | 100 | 100 | 100 | 94.4% |
| Robson) *^{d} | (92.3-100%) | (84.6-100%) | (84.6-100%) | (92.3-100%) | |
| MD *^{e} | 88.9 | 94.4 | 91.6 | 88.9 | 84% |
| | (44.4-100%) | (55.6-100%) | (77.8-100%) | (55.6-100%) | |
| | [3] | [3] | [3] | [1] | |
| OFM *^{e} | 61.7 | 60.5 | 63 | 60 | 59.5% |
| | (26.7-98.3%) | (35-100%) | (28.3-96.7%) | (43.3-96.7%) | |
| | | | [1] | | |
| OCT (μm) | | | 100.8 | 87.5 | 100.1*^{g} |
| | | | (80.8-116.4) | (54.2-106.1) | |
| | | | [3] | p = 0.046 [1] | |
| VEP | 4.4 | | 5.3 | 5.2 | |
| amplitude | (2.6-12.5) | | (1.2-14.3) | (1.2-7) | |
| (μV) | | | | | |
| VEP | 119 | | 125 | 118 | 103.8*^{h} |
| latency(ms) | (105-134) | | (115-139) | (115-138) | |
| | p = 0.0004 | | p = 2*10⁻⁶ | p = 5*10⁻⁵ | |

Remarks for Tables 2 and 3:
*a) In units of Decimal. Normal range is according to the Ranges of Vision Loss by the International Council of Ophthalmology (Resolution Adopted by the International Council of Ophthalmology. Sydney, Australia, Apr. 20, 2002. Acuities expressed as the percentage from optimal vision are given below. Optimal vision was defined for this purpose as 1 decimal (acuities ≧1 decimal were considered as 100%).
*b) The percentile of the field detected (i.e. points in the visual field detected at above a chance level: more than 15 out of 30 stimulations [22]. Similar results were also obtained when testing the whole visual field (0-24°).
*c) The percentile of correct responses (out of the total of 10 items in the test).
*d) In units of log MAR. CS expressed as the percentage from optimal vision (1.95) are given below.
*e) Motion detection (MD) and OFM tests: The percentiles of correct responses are given. Normal mean was defined as the mean performance level of the matched control subjects. Number of participants with missing data, when applicable, is given in squared parentheses at the bottom of each cell.
*f) Mantyjarvi M, Laitinen T. Normal values for the Pelli-Robson contrast sensitivity test. J Cataract Refract Surg 2001; 27(2):261-266.
*g) Budenz D L, Anderson D R, Varma R, et al. Determinants of normal retinal nerve fiber layer thickness measured by Stratus OCT. Ophthalmology 2007; 114(6):1046-1052.
*h) Halliday A M, McDonald W I, Mushin J. Visual evoked response in diagnosis of multiple sclerosis. Br Med J 1973; 4(5893):661-664.
p values denote significant differences in comparison to optimal vision level (in visual field and color perception measures) and in comparison to the normal population mean (for visual acuity, contrast sensitivity, OCT and VEP measures). p values in the OFM and motion detection tests denote significant differences in comparison to the mean of the matched control subjects. p values in the VEP amplitude (AE/HE) denote significant differences from 100%. Normal values are indicated in the right column.

### (d) Functional MRI

During an MRI scan, several tasks were performed, including (i) viewing flickering checkerboard for activating primary visual regions (V1); (ii) viewing an expanding-contracting array of dots, for activating the motion-related higher visual area (middle-temporal, MT); (iii) static object recognition: subjects viewed objects whose contours are defined by luminance differences and were asked to covertly name them, this stimulus activates the object-related higher visual area (lateral occipital cortex, LOC); and (iv) OFM extraction: subjects viewed motion-defined objects presented at two speeds (0.25 degrees/s and 2 degrees/sec), and were asked to press a response button when they recognized the object and to covertly name it. Blocks of coherent moving noise (presented at either slow or fast velocities) were also presented. As in the behavioral tests, only the OFM at the low speeds were included in the data analysis. The experiment was conducted using a block design paradigm. All experimental epochs lasted 12 seconds followed by 9 seconds of rest period. The rest condition served as a hemodynamic baseline condition. The subjects were required to fixate in the center of the screen during all tasks. All experimental conditions were presented in a monocular display. The order of stimulating the eyes was counterbalanced across subjects.

The Blood Oxygenated Level Dependent (BOLD) fMRI measurements were performed in a whole-body 3T, Siemens Trio scanner. The functional MRI protocols were based on a multi-slice gradient echo-planar imaging and a standard head coil. The functional data were obtained under the optimal timing parameters: T_{R}=3s, T_{E}=30 ms, flip angle=90°, imaging matrix=80*80, FOV=220 mm. The 33 slices with slice thickness of 3 mm (with 1 mm gap) were oriented in the axial position. The scan covered the whole brain.

Before statistical analysis, head motion correction, slice scan time correction and high-pass temporal smoothing in the frequency domain were applied to remove drifts and to improve the signal-to-noise ratio. Spatial smoothing (spatial Gaussian smoothing, FWHM=8 mm) were also obtained. A general linear model (GLM) approach was used to generate statistical parametric maps (modeling the hemodynamic response function using parameters as in [Boynton G M, Engel S A, Glover G H, Heeger D J. Linear systems analysis of functional magnetic resonance imaging in human V1. J Neurosci 1996; 16:4207-4221]).

Across-subject statistical parametric maps were calculated using a hierarchical random effects model [Friston K J, Holmes A P, Price C J, Buchel C, Worsley K J. Multi subject fMRI studies and conjunction analyses. Neuroimage 1999; 10:385-396], allowing a generalization of the results to the population level. This was done after the voxel activation time courses of all subjects were transformed into Talairach space[Talairach J, Tournoux P. Co-planar stereotaxic atlas of the human brain. New York: Thieme; 1988], Z-normalized and concatenated.

Significance levels were calculated taking into account the probability of a false detection for any given cluster by a Monte-Carlo simulation approach [Forman S D, Cohen J D, Fitzgerald M, Eddy W F, Mintun M A, Noll D C. Improved assessment of significant activation in functional magnetic resonance imaging (fMRI): use of a cluster-size threshold. Magn Reson Med 1995; 33(5):636-647] (1,000 iterations), extended to 3D data set cortical voxels using the threshold size plug-in in BrainVoyager Q X.

Three Regions Of Interest (ROIs) within the visual cortex were defined. Voxels in the V1 ROI were collected according to an anatomical marker: the calcarine sulcus including its upper and lower banks. Voxels in the object-related and motion-related regions (LOC and MT ROIs) were collected according to external functional localizers. Two separate experiments, designed in order to functionally localize the object-related and motion-related visual cortices, were performed in each subject. A binocular stimulation was used during in the functional localizers' scans.

The object-related area in the lateral occipital complex (LOC) localizer was composed of 2 conditions in a conventional block design apparatus; blocks of objects and blocks of scramble versions of these objects counterbalanced. Regions which were greatly activated by the objects in comparison to their scramble versions were defined as the LOC ROI [Malach R, Reppas J B, Benson R R, et al. Object-related activity revealed by functional magnetic resonance imaging in human occipital cortex. Proc Natl Acad Sci USA. 1995; 92:8135-8139].

The motion related area (MT) localizer was composed of 2 conditions: moving and stationary low contrast rings within a conventional block-design apparatus [Tootell R B, Reppas J B, Kwong K K, et al. Functional analysis of human MT and related visual cortical areas using magnetic resonance imaging. J Neurosci 1995; 15:3215-3230]. Regions which were greatly activated by the moving in comparison to the stationary rings were defined as the MT ROI. Anatomical and functional borders were taken into account when defining the LOC and MT localizers.

The individual activation level in each subject, assessed as Beta weights, was calculated in each ROI. Activation levels were then averaged across subjects.

### Results

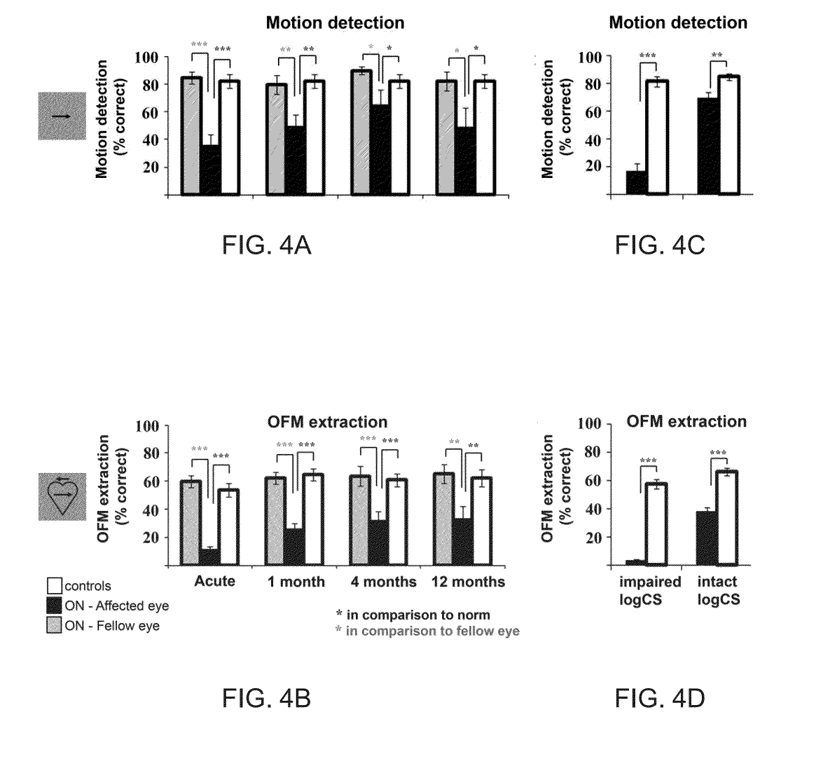

FIGS. 4A-D show sustained deficit in motion perception; independent of contrast sensitivity levels. Performance levels in the two motion related tasks; Motion detection (FIG. 4A) and OFM extraction (FIG. 4B) during the four testing phases. FIG. 4C shows motion detection, and FIG. 4D shows OFM extraction results for the AEs when grouped according to their CS levels (averaged across all testing phases). FIGS. 4A-D plot motion perception levels of AEs with impaired and intact CS, as compared to their matched control subjects. White bars are matched control subjects, black bars are affected eyes (AE) of ON patients, gray bars are fellow eyes (FE) of ON patients. Asterisks denote significance level: *p<0.05; **p<0.01; ***p<0.001.

In the AEs—visual acuities, visual field and color perception were significantly impaired at the acute phase and recovered completely after one month. Contrast sensitivity displayed a longer deficit and recovered completely after 4 months (see detailed information in Table 2). In the FEs, all visual functions were within the normal range at all time phases (see detailed information in Table 3). While the group as a whole had recovered at the 12 month phase, two patients had a sustained severe visual impairment.

At the 12 month phase, the RNFL thickness of both eyes was reduced when compared to the normal mean [16], but was within the normal range (Table 2 and Table 3).

The VEP amplitudes of the AEs were increased between the acute and 4 months phases, but not subsequently. Both affected and fellow eyes had significantly prolonged VEP latencies at all testing phases (Table 2 and Table 3).

Thus, according to the observation made by the present Inventors, the routine visual functions 4 months following the acute episode are normal, yet VEP latencies are prolonged.

Improvement occurred in both routine visual tests and motion perception. This was evident up to the 4 months phase (p<0.05, paired T Tests between phases), but not subsequently. However, improvement was disparate across measures, as revealed by a eye*test*time interaction (F=2.44, p=0.045 repeated measures three-ways ANOVA).

Unlike the routine visual tests, motion perception was impaired during the entire follow-up period (Table 2 and FIGS. 4A-B). The AEs were impaired in both motion detection and OFM extraction tasks during all testing phases, in comparison to the normal mean of the matched-control subjects and to the FEs.

Without wishing to be bound to any particular theory, t is assumed that the sustained deficit in motion processing can be resulted from the combination of two factors.

Firstly, a disproportionate deficit in the acute episode was found in motion perception when compared to the other visual functions (e.g. p=5*10⁻⁵ and p=4*10⁻⁵ for comparison of OFM with VA and CS, paired T Tests). All measures were represented in a percent correct scale).

Secondly, there was less recovery of motion processing in comparison to the other visual measures. Thus, the OFM recovery level (defined as the deltas between the acute and 4 months phases) was lower in comparison to the recovery of VA or CS functions (p=0.03, p=0.045 respectively, paired T Test between deltas).

While direct relationship between the severity of visual impairment during the acute episode and the severity of impairment later in the disease, was not observed, severity of impairment in later phases (e.g., 12 months) was correlated with severity at the 1 month phase (linear least-squares regression with calculation of the correlation coefficient, r=0.93, 0.97 and 0.91 for VA, CS and OFM; p<10⁻⁴ in all).

Thus, according to the observation made by the present Inventors, the motion perception in the affected eye is impaired 1 year following the acute episode.

In order to assess the relation between the motion perception deficit and the impaired CS in the AE, all the AEs were separated into two groups according to their CS levels: eyes with intact (>1.6) and impaired (<1.6) CS. FIGS. 4C and 4D show the motion perception functions in the two groups compared to their matched-control subjects. Both groups of AEs with impaired or intact CS levels exhibited a deficit in motion perception tasks. Analysis of covariance (ANCOVA) revealed that the effect of group (AE vs. matched controls) was significant after taking into account CS levels of the AEs. Thus, according to the observation made by the present Inventors, motion perception deficit is independent of CS levels (F=157.3, p<0.001; F=165.7, p<0.001 for OFM and motion detection respectively).

fMRI studies were performed on a sub-group of 13 patients and their matched-control subjects. The patient sub-group was indistinguishable from the whole group of patients in all visual functions, VEP and OCT measures. This was true for both AEs and FEs during all testing phases (two-sample T Tests, p>0.3 in all comparisons).

During the fMRI scan, subjects viewed flickering checkerboard, static objects, or an expanding-contracting array of dots. These stimuli activate the primary visual cortex (V1), the object-related region (LOC) and motion-related region (MT), respectively.

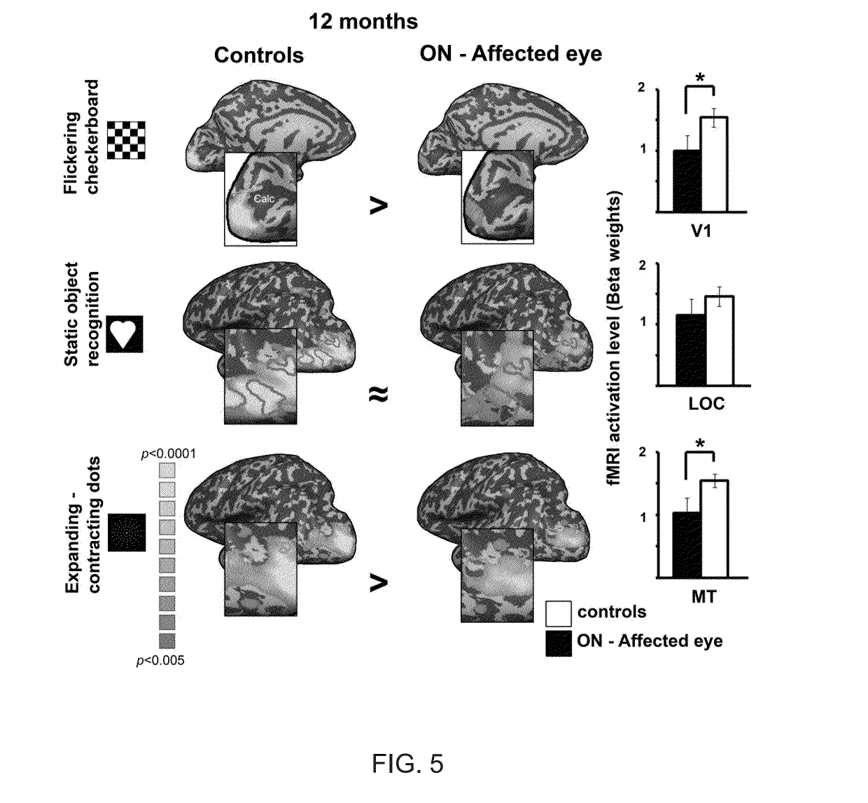

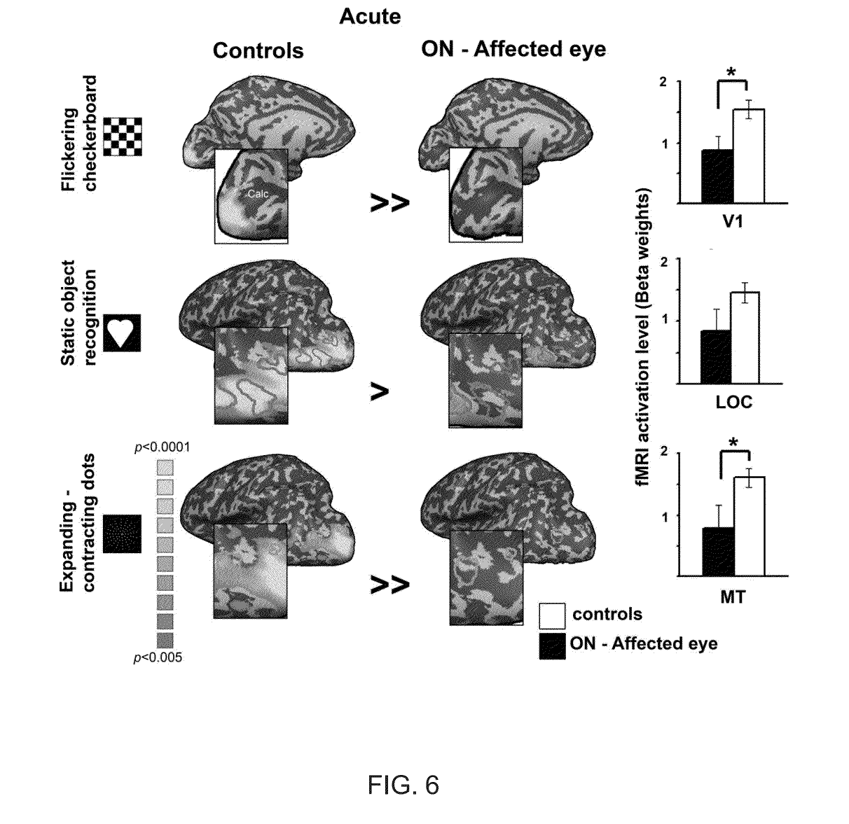

FIGS. 5 and 6 show fMRI activation maps which describe activation within the V1, LOC and MT regions while viewing of flickering checkerboard (top row), static objects (middle row) and expanding-contracting dots (bottom row), 12 months following the acute phase (FIG. 5) and during the acute phase (FIG. 6). The data are presented on a full Talairach normalized inflated brain of the left hemisphere. V1 is anatomically defined in the Calcarine sulcus (Calc), presented on a medial view of the cortex (upper raw). LOC and MT are outlined on the lateral view of the cortex (LOC is presented in purple lines, MT is presented in green lines, second and third rows correspondingly). Blow-ups highlight activation in the 3 ROIs. Activation above p=0.005 (corrected for multiple comparison) is presented; color scale denotes significance levels. Activation is shown for control subjects and affected eyes of ON patients. Histograms on the right denote the activation levels (beta weights) within each ROI for the two groups.

As shown in FIG. 5, viewing static objects elicited robust activation in LOC in ON patients and controls. While activation was slightly reduced during AE stimulation, a major part of LOC was activated. Viewing moving stimuli via the AE elicited activation only in a small part of MT. This co-occurs with the reduced activation in V1 during checkerboard presentation to the AE. In addition to the multi subjects' cortical activation maps, the fMRI activation levels were quantitatively assessed on a subject-by-subject basis. Activation levels were measured as the beta weights in the three ROIs: V1, MT and LOC. Reduced activation for the AE, as compared to controls, was observed in V1 and MT but not in LOC.

During the acute phase, a considerable activation reduction relative to controls is shown for AE stimulation in all three ROIs. Viewing static objects elicited some activation in LOC in all ON patients. MT was not consistently activated across patients (thus no activation is seen in the group random effect analysis map). The beta weights plots demonstrate that a significant reduction in fMRI activation is found in V1 and MT but not LOC

Thus, according to the observation made by the present Inventors, cortical activation associated with motion perception is reduced 1 year following the acute episode.

In order to address the neuronal basis of the behavioral OFM task, an fMRI using this same paradigm was performed. Subjects viewed either luminance or motion-defined objects (OFM). If patients experience a specific deficit in motion perception, reduced cortical activation is seen only for the second stimulus type, since motion perception is required to recognize OFM but not luminance-defined objects. Since OFM combines both motion and object perception, this stimulus is expected to activate both MT and LOC (in addition to primary visual cortex).

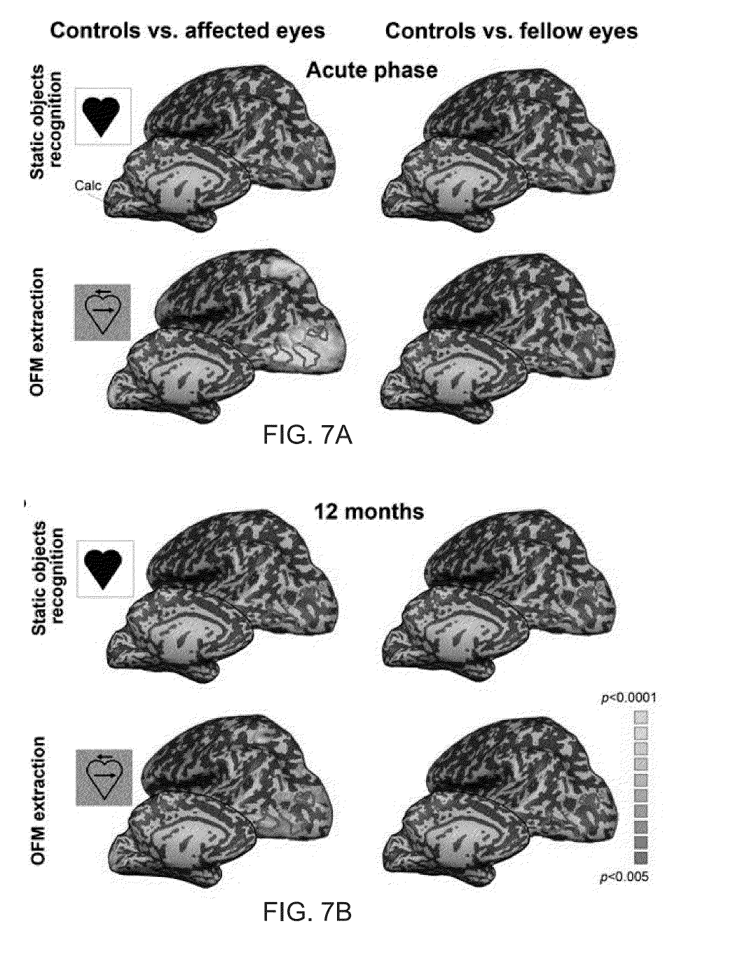

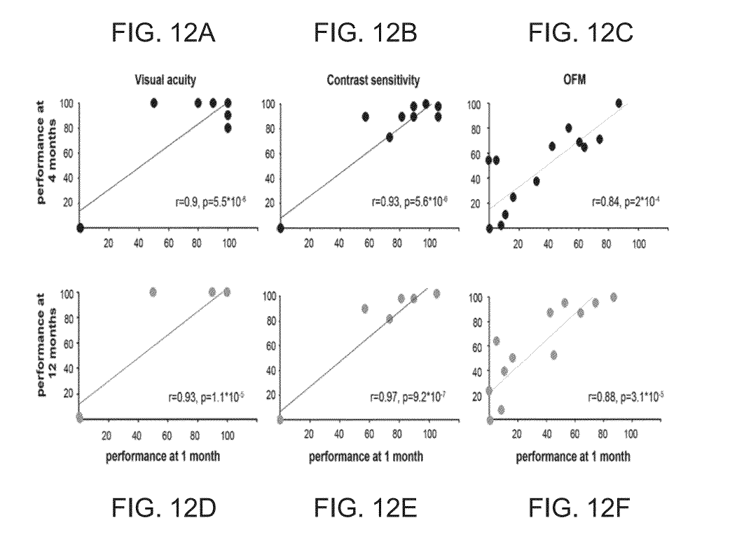

FIGS. 7A-B are differential fMRI activation maps showing cortical activation for controls versus ON patients (controls >ON patients) during static object and OFM viewing. The cortical activation obtained during AE and FE stimulation (in comparison to controls) is shown for the acute (FIG. 7A) and 12 months (FIG. 7B) phases. The data are presented on a full Talairach normalized inflated brain of the left hemisphere. Lateral and medial views (upper and lower views for each stimulation) are shown. Activation above p=0.005 (corrected for multiple comparison) is presented; color scale denotes significance levels. FIGS. 7A-B plot regions which have increased activation in the matched control subjects in comparison to the affected or fellow eyes of the ON patients (left and right columns respectively). ROIs are outlined as in FIG. 5.

The differential activation maps highlight voxels with greater activation in the controls compared with the ON group. The cortical activation levels obtained when subjects viewed static objects via the AE, were not different than those obtained in controls. This was found in all testing phases, including the acute phase. In comparison, viewing OFM stimuli via the AE resulted in robust differential cortical activation in various occipital regions including V1, LOC and MT (differential activation is also seen in sensorimotor regions since subjects were instructed to press a response button when they identified the OFM). A reduced cortical activation while processing OFM stimuli was found as long as twelve months following the acute phase, indicating the sustained impairment in motion processing. fMRI activation patterns 4 months following the acute phase were similar to those obtained at the 12 month phase (data not shown).

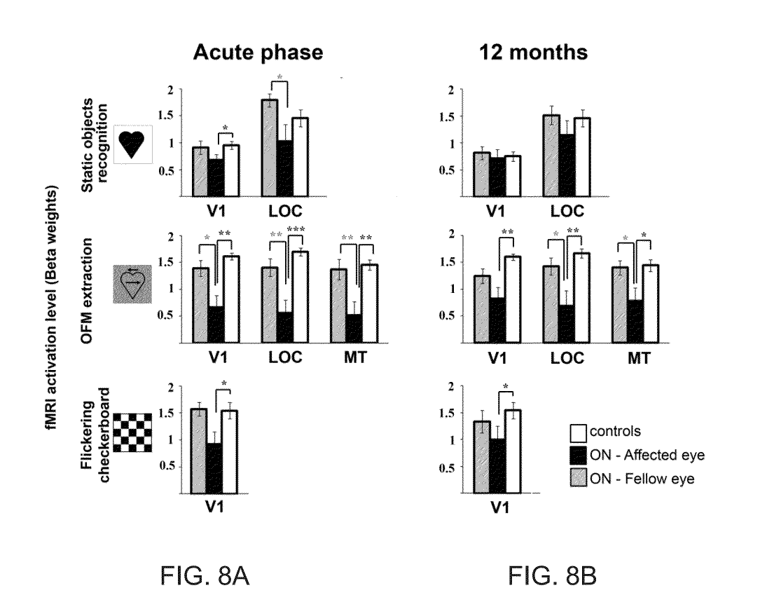

FIGS. 8A-B show fMRI activation levels (beta weights) during viewing of static objects (top row), OFM (middle row), and flickering checkerboard (bottom row) in the 3 ROIs: V1, LOC and MT, during the acute (FIG. 8A) and 12 months (FIG. 8B) phases. Grayscales and asterisks as in FIG. 4. As shown, reduced activation during static object viewing occurred during the acute phase only. Reduced activation levels during OFM processing occurred in all ROIs at 12 months phase.

The results in the acute phase, as demonstrated in FIGS. 7 and 8, indicate that while some patients demonstrated reduced cortical activation during static objects processing (reduced averaged beta weights in V1, FIG. 8), this is not a general phenomenon and thus does not survive the random effect model (FIG. 7). Reduced activation during dynamic object processing, on the other hand, is common to all patients and can be generalized to the ON population level.

Thus, the cortical activation for motion-defined objects verified the psychophysical findings.

### Discussion

The present Example provides evidence for a sustained motion perception deficit following ON, while static visual functions recovered. This effect was demonstrated using novel tests developed by the present Inventors. The deficit was evaluated relative to a group of 21 control subjects. The behavioral deficit in motion perception was associated with reduced cortical activation during motion processing. This was evident using different kinds of motion-related stimulation and different data analyses.

Previous longitudinal studies suggested that measures of low-contrast vision may be the most sensitive markers of visual dysfunction following ON [17-19]. The present Inventors found that CS continued to be impaired in comparison to visual acuity, visual field and color perception.

However, the motion perception test of the present embodiments revealed the most significant and prolonged impairment. Furthermore, the motion perception deficit was independent of CS levels.

The present Example demonstrated the advantage of the motion perception of the present embodiments in ophthalmologic tests following ON.

### REFERENCES

[1] Beck R W, Cleary P A, Backlund J C. The course of visual recovery after optic neuritis. Experience of the Optic Neuritis Treatment Trial. Ophthalmology 1994; 101(11):1771-1778.
[2] Cleary P A, Beck R W, Bourque L B, Backlund J C, Miskala P H. Visual symptoms after optic neuritis. Results from the Optic Neuritis Treatment Trial. J Neuroophthalmol 1997; 17(1):18-23; quiz 24-18.
[3] Grimsdale H. A Note on Pulfrich's Phenomenon with a Suggestion on Its Possible Clinical Importance. Br J Ophthalmol 1925; 9(2):63-65.
[4] Frisen L, Hoyt W F, Bird A C, Weale R A. Diagnostic uses of the Pulfrich phenomenon. Lancet 1973; 2(7825):385-386.
[5] Regan D, Kothe A C, Sharpe J A. Recognition of motion-defined shapes in patients with multiple sclerosis and optic neuritis. Brain 1991; 114 (Pt 3):1129-1155.
[6] Barton J J, Rizzo M. Motion perception in optic neuropathy. Neurology 1994; 44(2):273-278.
[7] Born R T, Bradley D C. Structure and function of visual area MT. Annual review of neuroscience 2005; 28:157-189.
[8] Werring D J, Bullmore E T, Toosy A T, et al. Recovery from optic neuritis is associated with a change in the distribution of cerebral response to visual stimulation: a functional magnetic resonance imaging study. Journal of neurology, neurosurgery, and psychiatry 2000; 68 (4): 441-449.
[9] Toosy A T, Hickman S J, Miszkiel K A, et al. Adaptive cortical plasticity in higher visual areas after acute optic neuritis. Annals of neurology 2005; 57(5):622-633.
[10] Levin N, Orlov T, Dotan S, Zohary E. Normal and abnormal fMRI activation patterns in the visual cortex after recovery from optic neuritis. Neuroimage 2006; 33(4):1161-1168.
[11] Korsholm K, Madsen K H, Frederiksen J L, Skimminge A, Lund T E. Recovery from optic neuritis: an ROI-based analysis of LGN and visual cortical areas. Brain 2007; 130(Pt 5):1244-1253.
[12] Jenkins T M, Toosy A T, Ciccarelli O, et al. Neuroplasticity predicts outcome of optic neuritis independent of tissue damage. Annals of neurology; 67(1):99-113.
[13] Jones S J, Brusa A. Neurophysiological evidence for long-term repair of MS lesions: implications for axon protection. Journal of the neurological sciences 2003; 206(2):193-198.
[14] Forman S D, Cohen J D, Fitzgerald M, Eddy W F, Mintun M A, Noll D C. Improved assessment of significant activation in functional magnetic resonance imaging (fMRI): use of a cluster-size threshold. Magn Reson Med 1995; 33(5):636-647.
[15] Friston K J, Holmes A P, Price C J, Buchel C, Worsley K J. Multisubject fMRI studies and conjunction analyses. Neuroimage 1999; 10(4):385-396.
[16] Budenz D L, Anderson D R, Varma R, et al. Determinants of normal retinal nerve fiber layer thickness measured by Stratus OCT. Ophthalmology 2007; 114(6):1046-1052.
[17] Trobe J D, Beck R W, Moke P S, Cleary P A. Contrast sensitivity and other vision tests in the optic neuritis treatment trial. American journal of ophthalmology 1996; 121(5):547-553.
[18] Balcer L J, Baier M L, Pelak V S, et al. New low-contrast vision charts: reliability and test characteristics in patients with multiple sclerosis. Multiple sclerosis (Houndmills, Basingstoke, England) 2000; 6(3):163-171.
[19] Mowry E M, Loguidice M J, Daniels A B, et al. Vision related quality of life in multiple sclerosis: correlation with new measures of low and high contrast letter acuity. Journal of neurology, neurosurgery, and psychiatry 2009; 80(7):767-772.
[20] Mantyjarvi M, Laitinen T. Normal values for the Pelli-Robson contrast sensitivity test. J Cataract Refract Surg 2001; 27(2):261-266.
[21] Halliday A M, McDonald W I, Mushin J. Visual evoked response in diagnosis of multiple sclerosis. Br Med J 1973; 4(5893):661-664.
[22] Chokron S, Perez C, Obadia M, Gaudry I, Laloum L, Gout O. From blindsight to sight: cognitive rehabilitation of visual field defects. Restorative neurology and neuroscience 2008; 26(4-5):305-320.

### Example 2

Example 1 above demonstrated a specific sustained deficit in dynamic visual functions following ON. The present Example describes a study directed to identify the mechanism of this deficit. In the current study, patients were followed-up longitudinally after an ON attack providing the timeline for recovery and visual outcome predictability.

### Methods

Twenty-one patients aged 18-59 (mean±STDEV 29±9.5) years presenting with a first-ever episode of acute ON participated in the study. Patients were enrolled during hospitalization. All patients presented with unilateral visual loss, a relative afferent pupillary defect, and an otherwise normal neuro-ophthalmological examination. Two patients had a recurrent attack during study follow-up, and their data were therefore excluded from subsequent analyses. Twenty-one control subjects who were matched to the patients for age, gender and dominant eye on a subject-by-subject-basis were included in the study. The Hadassah Hebrew University Medical Center Ethics Committee approved the experimental procedure. Written informed consent was obtained from all subjects.

Static visual function tests included visual acuity (VA, measured by Snellen visual-acuity chart), visual fields estimation (by the automatic Humphrey's perimetry visual-field test 24-2), color perception (Standard pseudoisochromatic plates, by Ichikawa) and contrast sensitivity (CS, Pelli-Robson chart at 1 meter, Metropia Ltd., Cambridge, UK).

Dynamic visual function tests included OFM extraction test, static objects test and coherent moving noise test.

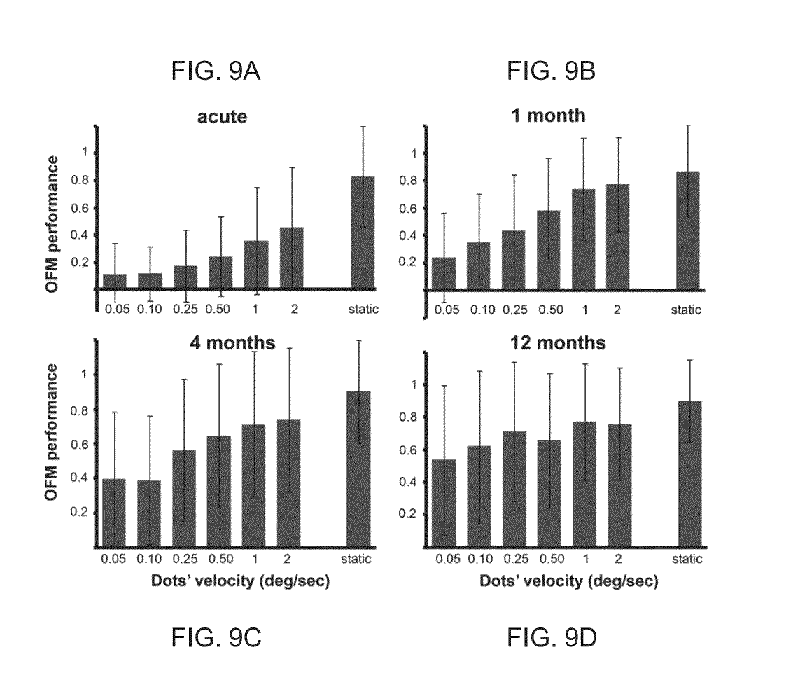

The OFM extraction test included OFM stimuli an object as further detailed hereinabove. The OFM stimuli were moved at six different speeds: 0.05, 0.1, 0.25, 0.5, 1 and 2 degrees/s. Only the lower 3 velocities were included in the data analysis, due to their increased sensitivity in detecting the motion perception deficit following ON (FIG. 9).

In the static objects, objects of which the contours are defined by luminance difference (white objects on a black background) were presented in order to rule out a naming bias which may interfere with the results of the OFM extraction task.

In the coherent moving noise test, arrays of dots (similar to the ones used for the OFM stimuli) were moved as a whole. Consequently motion but no object was apparent. These were presented as “foil trails”.

Stimuli were presented on a computer screen situated at a distance of about 50 cm from the subjects' eyes in a random order, each preceded by a 980 ms long fixation and lasting until the subject responded or for a maximum of 4 seconds. Fixation was not maintained during testing to avoid the impact of partial field defects on patients' performance level (this was especially relevant soon after the ON attack).

To avoid between-eye and between-phase learning, 4 experimental blocks were created, each consisting of 60 OFM stimuli (20 at each velocity), 12 moving noise stimuli (4 at each velocity) and 10 static objects. The two eyes of a subject were shown different blocks on each run, and each eye was shown different blocks on adjacent runs. The exact experimental block (1-4) presented to a patient was also shown to his control subject, matched on the basis of the tested eye. The experimental block shown to the dominant eye of a patient was also presented to the dominant eye of the matched control subject. This was done in each testing phase.

Visual tests were evaluated monocularly at the acute phase (during hospitalization, 3-5 days following hospital admission) as well as 1, 4 and 12 months following the attack.

Standard clinical lighting was used for visual testing.

The VEP amplitudes and VEP latencies of the major positive component (P100) were recorded on pattern reversal full-field checkerboards on a Bravo VEP device (Nicolet, Biomedical). Standard pattern reversal VEP parameters were used to enable the generalization of the results to other clinical centers. Achromatic checks were presented on a computer monitor screen, each check subtending 60′ at the eye. The screen subtended about 17° horizontal X 14° vertical. Two lateral electrodes were placed at O₁ and O₂ and were referenced to Fz. The ground electrode was positioned on the forehead. VEP latencies and amplitudes were assessed from either O₁ or O₂, selecting the electrode which produced the sharper VEP wave. This was exclusively chosen by the technician. In most cases, the waves obtained from O₁ and O₂ were similar. At least two repetitions were recorded for each eye, and the reported values are the average of these recordings.

Due to the wide range of variability within the normal population, to best study the effect of ON over time, VEP amplitudes from the affected eye (AE) were expressed as a percentage of that from the fellow eye (FE) [4]. Patients in whom the VEP waveform was unobtainable due to poor vision were excluded from the VEP latency analyses (n=7 in the acute phase, and n=2 in later phases). VEP was assessed monocularly at the acute, 4 and 12 month phases.

Patients' performance levels were expressed as a percentage of the normal values (norms) in each measure. Norms in the standard (static) visual tests were based on established norms that are available from the literature. These were defined as the mean normal population values (Snellen V A≧1 [1]; Pelli-Robson, logCS≧1.84 [14]). Dynamic visual function's (OFM) norms were based on the control group and were defined as the mean control subjects' values.

For each patient a delta score which represents the distance from normal level was calculated. Using two tailed T-test, significant difference was defined as having deltas that were significantly different from zero.

### Results

FIGS. 9A-D show performance levels in the different OFM speeds (referred to as dot's velocity in FIGS. 9A-D), for the acute (FIG. 9A), 1 month (FIG. 9B), 4 months (FIG. 9C) and 12 months (FIG. 9D) phases. Performance levels in the OFM task are plotted as a function of dots' speed (ranging from 0.05 to 2 degrees/s). The right column indicates performance for static, luminance-defined objects. Performance level of the optic neuritis eyes is expressed as a percentage of normal values (i.e. 100% performance level equals control subjects' mean, see methods).

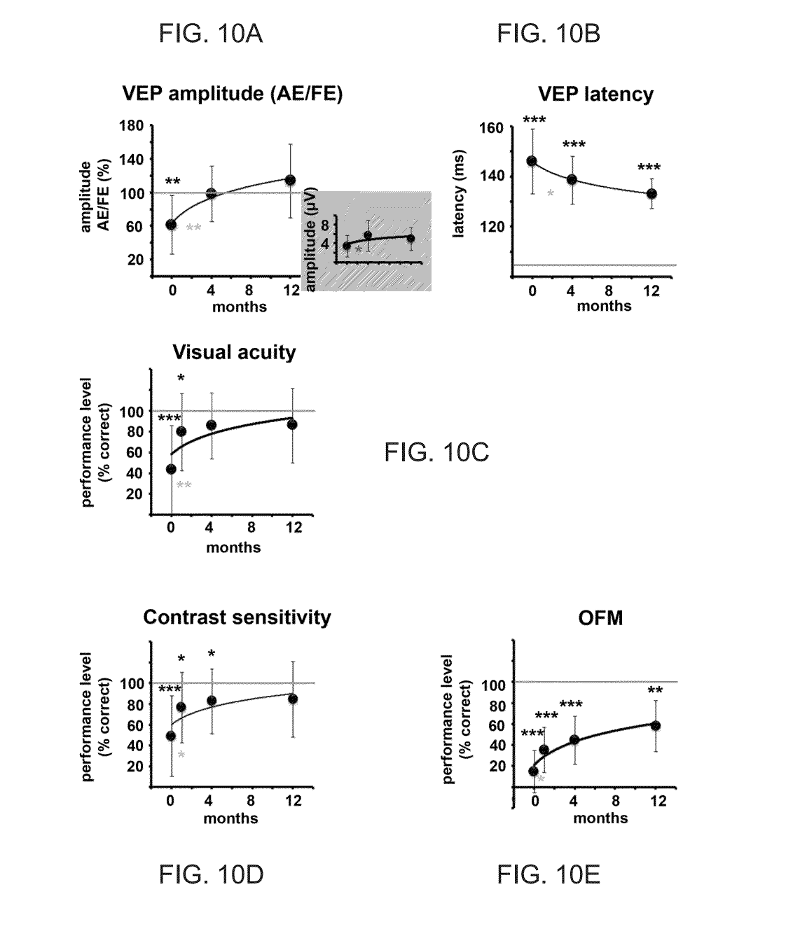

FIGS. 10A-E show VEP measurements, static and dynamic visual functions throughout the 12 month follow-up.

FIGS. 10A and 10B show changes in VEP amplitudes (FIG. 10A) and VEP latencies (FIG. 10B) over time. VEP amplitudes from the affected eye (AE) are expressed as a percentage of the fellow eye (FE). Inset denotes absolute amplitude levels for the AEs.

FIGS. 10C-E show changes in static visual functions. Visual acuity (FIG. 10C) and contrast sensitivity (FIG. 10D) and dynamic visual function (motion perception assessed by the OFM test, FIG. 10E) are plotted over time.

To obtain a direct comparison between measures, performance levels are expressed as a percentage of the normal values in each measure (100% of VA equals 1 decimal, 100% of CS equals log MAR=1.84, 100% of OFM equals control subjects' mean in each phase). Gray horizontal lines mark the mean normal values. N=21, 20, 18 and 14 in the acute, one, four and twelve months time points respectively. Black asterisks denote significant reduction of AEs' performance as compared to the normal values. Gray asterisks denote significant change in the AEs measurements between testing phases. A single asterisk symbol (*) denotes p<0.05, a two asterisk symbol (**) denotes p<0.01, and a three asterisk symbol (***) denotes p<0.001.

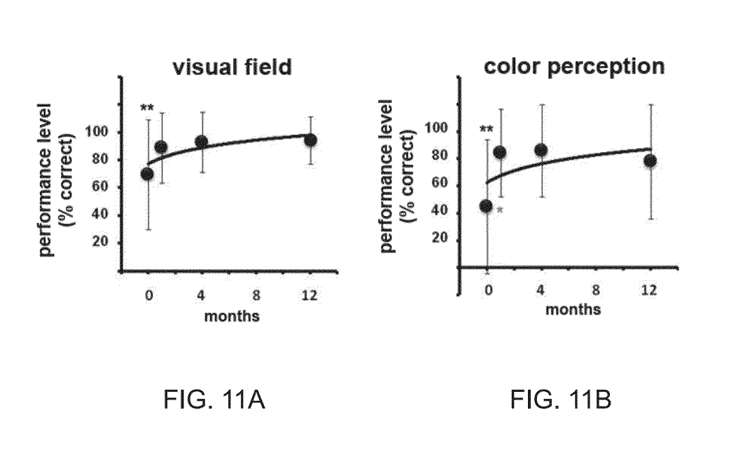

FIGS. 11A-B show static visual functions throughout the 12 month follow-up. Shown in FIGS. 11A and 11B are changes in visual field (FIG. 11A) and color perception (FIG. 11B) over time. N=21, 20, 18 and 14 in the acute, one, four and twelve months time points, respectively. Black asterisks denote significant reduction of the AEs' performance level as compared to the normal values. Gray asterisks denote significant change in the AEs measurements between testing phases. A single asterisk symbol (*) denotes p<0.05, a two asterisk symbol (**) denotes p<0.01, and a three asterisk symbol (***) denotes p<0.001.

VEP amplitudes of the AEs were significantly reduced compared to the FEs in the acute phase (p<0.01, 2-tailed T test). These differences disappeared in subsequent phases (FIG. 10A). VEP latencies of the AEs were significantly prolonged at all testing phases, when compared to the normal population's mean (103.8 ms [15]) or the normal range (115 ms [15], FIG. 10B).

Visual acuity (VA) was severely impaired at presentation, measuring 0.44 decimal (corresponding to about 20/45). However, by four months VA was not significantly different from 1 decimal (corresponding to 20/20) (FIG. 10C).

Visual field and color perception measurements were significantly impaired at the acute phase and recovered after 1 month (FIGS. 11A-B). Contrast sensitivity (CS) was significantly impaired during the first 4 months, when compared to the normal populations' mean (Pelli-Robson, log CS=1.84) [14], but subsequently recovered (FIG. 10D).

Unlike the static visual tests, the AEs demonstrated a sustained deficit in motion perception, as evident in the OFM extraction task 12 months following the acute phase. The maximum level reached by the AEs was less than 60% of the normal performance level (averaged across eyes, FIG. 10E).

Fellow eyes were not impaired in any visual tests, at any of the follow-up phases (data not shown in the current report).

Thus, according to the observation made by the present Inventors, 12 months following the acute phase, VEP latencies were prolonged, static visual functions returned to normal, while motion perception was impaired.

With respect to the visual outcomes on the group level, a significant improvement in both visual functions and VEP measures was evident only within the first 4 months. Changes between 4 and 12 months were not significant. After 4 months, VEP amplitudes recovered coinciding with recovery of VA and CS functions. Although VEP latency shortened significantly during the first 4 months, it remained significantly prolonged. Similarly, motion perception improved within the first 4 months, but remained impaired.

Motion perception was disproportionately impaired at baseline and recovered less than static visual functions. The rate of initial recovery was greater for static functions (improvement of 36, 27 and 20% in the first 1 month for VA, CS and OFM respectively), while the subsequent recovery rate was similar among the visual functions (2, 2.5 and 3% per month for VA, CS and OFM, respectively, up to the 4 month phase).

Thus, according to the observation made by the present Inventors, no significant visual or electrophysiological improvement was evident beyond 4 months following the acute phase.

FIGS. 12A-F show performance levels in visual acuity (FIGS. 12A and 12D), contrast sensitivity (FIGS. 12B and 12E) and OFM tasks (FIGS. 12C and 12F) at the 1 month time point, plotted against performance level assessed at the 4 month phase (FIGS. 12A-C) and 12 month phase (FIGS. 12D-F). Each symbol corresponds to one patient (N=18 and 14 in the 4 and 12 months phases respectively)

As shown, the visual outcome on a subject-by-subject basis reveals that visual performance 1 month following the attack is highly predictive of visual recovery. Visual performance at the 1 month time point was strongly correlated with visual performance at subsequent time points. This was seen for VA, CS and OFM functions (VA: r=0.9 p=5.5*10⁻⁶, CS: r=0.93 p=5.6*10⁻⁶, OFM: r=0.84 p=2*10⁻⁴ for correlating the 1 and 4 months time points. VA: r=0.93 p=1.1*10⁻⁵, CS: r=0.97 p=9.2*10⁻⁷, OFM: r=0.91 p=3.1*10⁻⁵ for correlating the 1 & 12 months time points).

The process of recovery of static and dynamic visual functions behaved according to different patterns. Regarding static functions, patients with VA greater than 0.4 at the 1 month phase (19/21 in the studied cohort) recovered completely, while those suffering from complete visual loss 1 month following the attack (2/21 patients) remained blind in their AE (for the duration of the 12 month follow-up phase). Similar findings were found for CS. Thus, the outcomes of static functions appear to follow an “all-or-none” pattern.

Regarding dynamic functions, the rate of improvement in the OFM task following the 1 month time point was similar across ON patients, regardless of their initial performance level (mean 12%, median 6% for improvement between the 1 and 4 month time points across patients' cohort. No correlation was found between initial OFM levels and improvement rate r=−0.1, p>0.05). Since patients improve at a constant rate, outcome is dependent on OFM levels at the 1 month time-point. Thus, according to the observation made by the present Inventors, visual outcome can be predicted 1 month following the attack

In order to study the effects of VEP amplitudes on visual functions, the eyes were separated into two groups corresponding to either intact or impaired amplitudes.

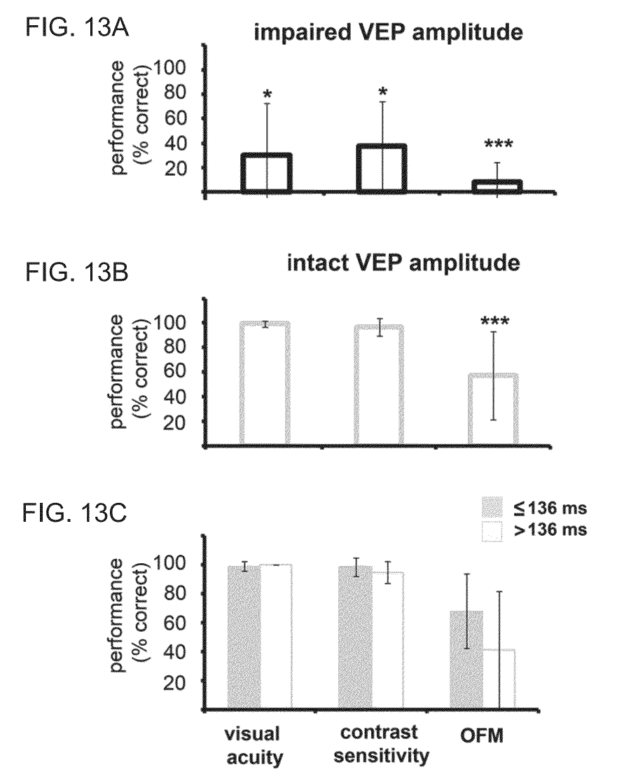

FIGS. 13A-C show visual measurements as a function of VEP amplitudes and latencies. FIGS. 13A and 13B show visual acuity, contrast sensitivity and OFM levels in optic neuritis eyes with impaired (FIG. 13A) and intact (FIG. 13B) amplitudes (n=5 and 16, correspondingly). FIG. 13C shows performance levels in the different visual measurements for eyes with intact amplitudes, divided according to their VEP latencies (shorter than 136 ms, n=8; or longer than 136 ms, n=8. A threshold of 136 ms was chosen since this was the median latency level among eyes with intact amplitudes). The data included for each patient is taken from his latest time point available. Asterisks denote significant reduction of AEs' performance as compared to the normal values, a single asterisk symbol (*) denotes p<0.05, a two asterisk symbol (**) denotes p<0.01, and a three asterisk symbol (***) denotes p<0.001.

As shown in FIG. 13A, impaired VEP amplitudes disturb various types of visual functioning, resulting in impaired static and dynamic functions. Intact VEP amplitudes (FIG. 13B) are associated with recovered VA and CS, suggesting that these visual functions depend solely on a sufficient amount of visual information reaching the cortex. Given that all ON eyes had prolonged VEP latencies, the data indicate that VA and CS do not relate to the latency of visual projection. However, motion perception was impaired even in patients with intact VEP amplitudes. This suggests that an intact amount of visual projection is insufficient for the completion of dynamic visual functions.

In order to investigate the effect of VEP latencies on motion perception, the eyes with intact VEP amplitudes were divided according to their projection rates (less the or equal 136 ms, and above 136 ms). An association between VEP latencies and OFM performance levels was found. Specifically, longer VEP latencies were associated with reduced motion perception levels (FIG. 13C).

Thus, according to the observation made by the present Inventors, VEP amplitudes can explain static but not dynamic visual functions.

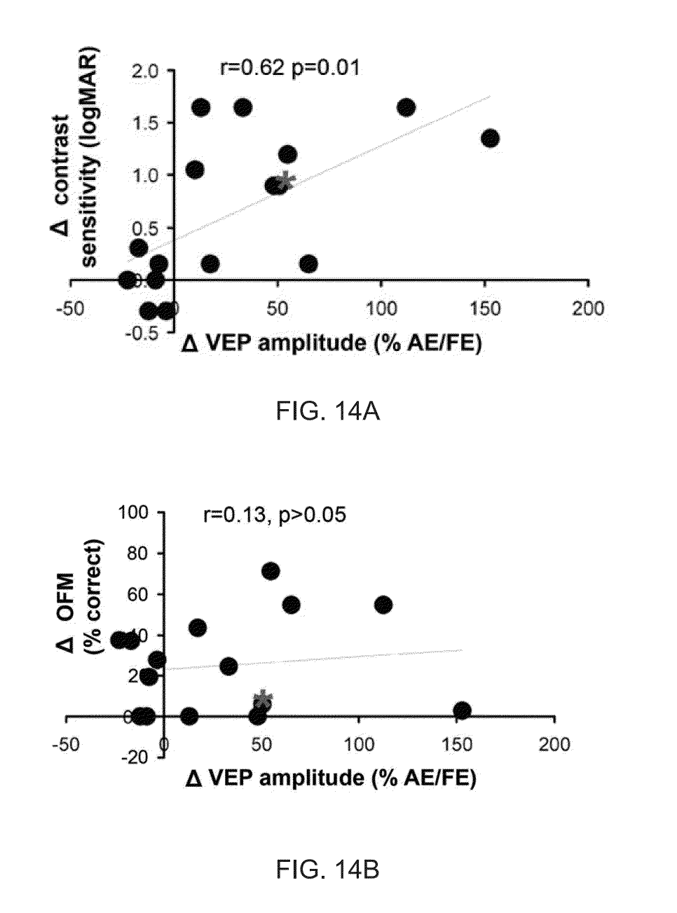

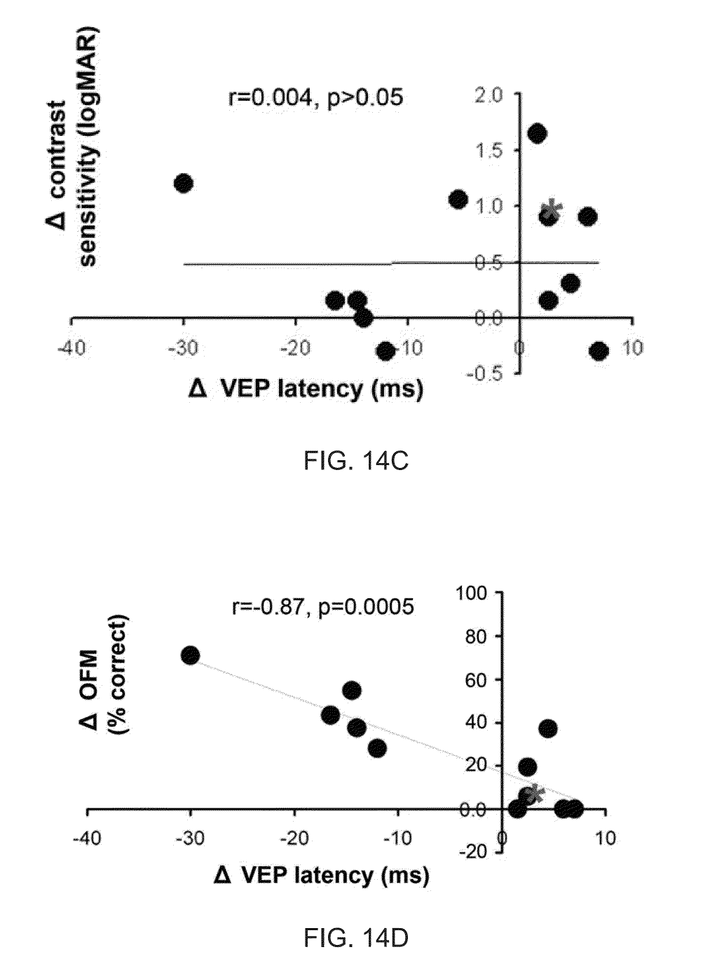

FIGS. 14A-D show correlation between the changes in VEP measurements and visual functions. Shown in FIGS. 14A-D are correlations between changes in VEP amplitudes (FIGS. 14A and 14B) or latencies (FIGS. 14C and 14D) and visual functions: contrast sensitivity (FIGS. 14A and 14C) and OFM (FIGS. 14B and 14D). Each symbol corresponds to one subject, indicating the delta between his acute and 4 month phases (4 months—acute scores).

Asterisks denote one specific patient. His data is marked to demonstrate the reliance of CS improvement on VEP amplitudes restoration and the insufficiency of this condition to accomplish dynamic visual functions: Patients' VEP amplitudes improved by 51% from the acute to the 4 month phase (from 45% to 96%) and his VEP latency was elongated by 2.5 ms (131 to 133.5 ms). Correspondingly, his CS function improved by 0.9 units of log MAR, while a minimal improvement of 6% was evident in his OFM performance level.

The phases shown in FIGS. 14A-D were selected since significant changes occurred only during this time period, see FIG. 10). An increase in VEP amplitudes was significantly correlated with improvement in CS, but not OFM levels (F=8.8; p=0.01; r=0.62 for CS and F=0.22; p>0.05; r=0.13 for OFM, FIGS. 14A-B). Shortening of VEP latencies was significantly correlated with improved OFM levels (linear least-squares regression with calculation of the correlation coefficient F=27.3; p=0.0005; r=−0.87) but not with CS dynamics (F=0.0002; p>0.05; r=0.004, FIGS. 14C-D). Thus, while the quantity of CS improvement relates to the amount of VEP amplitude restoration, the quantity of OFM improvement depends on the extent of VEP latency reduction. It is therefore concluded that VEP latency prolongation can explain the motion perception deficit

### Discussion

The reduction in VEP amplitudes, evident at the acute phase of ON resolved within four months, along with the recovery of standard visual functions. VEP latency prolongation was evident 12 months after the acute phase. This abnormality co-occurred with a sustained deficit in motion perception and a significant correlation was found between the two measures.

The window for recovery in all visual measures was only seen within the first 4 months after the ON attack. Moreover, patient's visual outcomes were already determined at the 1 month phase. In static functions, all patients who initiated the recovery process 1 month following the attack recovered in subsequent phases. In dynamic functions, the rate of recovery was constant across patients, irrespective of the initial deficit level.

VEP amplitude reduction caused by conduction block or axonal atrophy reflects an insufficient amount of visual input. This deafferentiation interferes with various types of visual functioning. Intact VEP amplitudes result in recovery of static visual functions (FIG. 13). VEP amplitudes were found to correlate with RNFL thickness levels (e.g. [8, 9]), and a high functional-topographic correlation was found between the two [16].

Delivery of a sufficient amount of visual information is a pre-requisite to accomplishing any visual task. The results obtained by the present Inventors demonstrate that this is true for both static and dynamic visual functions. However, restoration of VEP amplitudes alone is insufficient for the execution of dynamic visual functions (FIG. 13). Patients who had a significant improvement in their VEP amplitudes but minimal shortening of their prolonged latencies, demonstrated improvement in their CS function but not in their OFM performance level (FIG. 14, see the patient denoted by an asterisk).

Thus, according to the observation made by the present Inventors, Axonal integrity affects static visual functions.

VEP latency prolongation co-occurring with normal signal amplitude reflects an intact amount of visual information delivered with a time delay. The present study demonstrated that changes in VEP latencies during the first year following the attack are associated with changes in dynamic but not static visual functions. This suggests that the myelination status following ON can be evaluated by dynamic visual functions and their electrophysiological correlate, VEP latency.

Impaired processing of high frequency information following demyelination has been described in several sensory systems. In the somato-sensory domain, loss of vibration sensitivity is thought to be related to the inability of demyelinated nerve fibers to transmit rapid trains of impulses [10, 19]. In the visual domain, impaired temporal resolution of vision, delayed visual perception and motion perception deficit were reported in ON and MS patients [13, 20, 21, 22]. The results of the present study, which demonstrate a close relationship between motion perception and delayed VEP latencies, suggest that these deficits are caused by the inability of demyelinated optic nerve fibers to transmit high temporal frequency information. Thus, motion perception may provide a possible behavioral correlate for VEP latency prolongation. It is therefore concluded that demyelination processes may specifically affect temporal aspects of perception.

Given the strong correlation between shortening of VEP latencies and improvement in dynamic visual functions, the natural history of motion perception may reveal the progress of nerve myelin pathology following ON.

The magnitude of improvement in motion perception was found to be constant across patients independent of the initial deficit level, suggesting that remyelination processes have a constant rate, regardless of initial demyelination. This is in accordance with a previous report demonstrating that the magnitude of latency shortening was independent of initial latency delay (measured by multifocal VEP) [24]. The consistency in remyelination magnitude across patients may stem from the unique elongated optic nerve geometry, limiting the interaction between oligodendrocyte precursor cells and the demyelinated axons. This structural limitation overpowers other patient and lesion related factors, which are known to be important in the remyelination process [25]. The limited cross section area and the known limited time window open for remyelination [26, 27] can explain the lack of significant changes in VEP latencies and motion perception beyond 4 months after the attack.

Thus, the dynamic visual functions of the present embodiments can be used as a longitudinal marker of demyelination and remyelination processes in the visual pathways.

The results presented in this Example can be evaluated in the light of currently developing neuro-protective and regenerative therapeutic strategies, targeting myelination in the CNS. The combination of VEP studies and dynamic visual functions, which were found by the present Inventors to be correlated, can be used as non-invasive tools to follow processes of demyelination and remyelination in the visual pathways. The observation that the magnitude of remyelination (as expressed by OFM improvement) is independent of initial demyelination, may serve as a baseline to assess the efficacy of therapeutic strategies. Since the expected rate of recovery is constant across patients, the success of interventions can be ascertained.

### REFERENCES

[1] Halliday A M, McDonald W I, Mushin J. Delayed visual evoked response in optic neuritis. Lancet 1972; 1(7758):982-985.
[2] Halliday A M, McDonald W I, Mushin J. Delayed pattern-evoked responses in optic neuritis in relation to visual acuity. Transactions of the ophthalmological societies of the United Kingdom 1973; 93(0):315-324.
[3] K H C. Evoked Potentials in Clinical Medicine Third edition ed. New York: Lippincott Williams & Wilkins; 1977.
[4] Jones S J, Brusa A. Neurophysiological evidence for long-term repair of MS lesions: implications for axon protection. Journal of the neurological sciences 2003; 206(2):193-198.
[5] Plant G T. Transient visually evoked potentials to sinusoidal gratings in optic neuritis. Journal of neurology, neurosurgery, and psychiatry 1983; 46(12):1125-1133.
[6] Sanders E A, Volkers A C, van der Poel J C, van Lith G H. Visual function and pattern visual evoked response in optic neuritis. Br J Ophthalmol 1987; 71(8):602-608.
[7] Bodis-Wollner I, Hendley C D, Mylin L H, Thornton J. Visual evoked potentials and the visuogram in multiple sclerosis. Annals of neurology 1979; 5(1):40-47.
[8] Trip S A, Schlottmann P G, Jones S J, et al. Retinal nerve fiber layer axonal loss and visual dysfunction in optic neuritis. Annals of neurology 2005; 58(3):383-391.
[9] Klistorner A, Arvind H, Nguyen T, et al. Axonal loss and myelin in early ON loss in postacute optic neuritis. Annals of neurology 2008; 64(3):325-331.
[10] Halliday A M, McDonald W I. Pathophysiology of demyelinating disease. British medical bulletin 1977; 33(1):21-27.
[11] Thurtell M J, Bala E, Yaniglos S S, Rucker J C, Peachey N S, Leigh R J. Evaluation of optic neuropathy in multiple sclerosis using low-contrast visual evoked potentials. Neurology 2009; 73 (22): 1849-1857.
[12] Raz N, Dotan S, Benoliel T, Chokron S, Ben-Hur T, Levin N. Sustained motion perception deficit following optic neuritis: Behavioral and cortical evidence. Neurology 2011; 76(24):2103-2111.
[13] Regan D, Kothe A C, Sharpe J A. Recognition of motion-defined shapes in patients with multiple sclerosis and optic neuritis. Brain 1991; 114 (Pt 3):1129-1155.
[14] Mantyjarvi M, Laitinen T. Normal values for the Pelli-Robson contrast sensitivity test. J Cataract Refract Surg 2001; 27(2):261-266.
[15] Halliday A M, McDonald W I, Mushin J. Visual evoked response in diagnosis of multiple sclerosis. Br Med J 1973; 4(5893):661-664.
[16] Klistorner A, Arvind H, Nguyen T, et al. Multifocal VEP and OCT in optic neuritis: a topographical study of the structure-function relationship. Documenta ophthalmologica 2009; 118 (2): 129-137.
[17] Neima D, Regan D. Pattern visual evoked potentials and spatial vision in retrobulbar neuritis and multiple sclerosis. Arch Neurol 1984; 41(2):198-201.
[18] Brusa A, Jones S J, Kapoor R, Miller D H, Plant G T. Long-term recovery and fellow eye deterioration after optic neuritis, determined by serial visual evoked potentials. Journal of neurology 1999; 246(9):776-782.
[19] McDonald W I, Sears T A. The effects of experimental demyelination on conduction in the central nervous system. Brain 1970; 93(3):583-598.
[20] Galvin R J, Regan D, Heron J R. Impaired temporal resolution of vision after acute retrobulbar neuritis. Brain 1976; 99(2):255-268.
[21] Heron J R, Regan D, Milner B A. Delay in visual perception in unilateral optic atrophy after retrobulbar neuritis. Brain 1974; 97(1):69-78.
[22] Barton J J, Rizzo M. Motion perception in optic neuropathy. Neurology 1994; 44(2):273-278.
[23] Kolappan M, Henderson A P, Jenkins T M, et al. Assessing structure and function of the afferent visual pathway in multiple sclerosis and associated optic neuritis. Journal of neurology 2009; 256(3):305-319.
[24] Klistorner A, Arvind H, Garrick R, Yiannikas C, Paine M, Graham S L. Remyelination of optic nerve lesions: spatial and temporal factors. Multiple sclerosis (Houndmills, Basingstoke, England) 2010; 16(7):786-795.
[25] Franklin R J, French-Constant C. Remyelination in the CNS: from biology to therapy. Nature reviews 2008; 9(11):839-855.
[26] Blakemore W F, Chari D M, Gilson J M, Crang A J. Modelling large areas of demyelination in the rat reveals the potential and possible limitations of transplanted glial cells for remyelination in the CNS. Glia 2002; 38(2):155-168.
[27] Chari D M, Blakemore W F. Efficient recolonisation of progenitor-depleted areas of the CNS by adult oligodendrocyte progenitor cells. Glia 2002; 37(4):307-313.
[28] Taveggia C, Feltri M L, Wrabetz L. Signals to promote myelin formation and repair. Nature reviews; 6(5):276-287.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of diagnosis, comprising:
using a display device for presenting a motion perception test to a subject;
determining a subject response to said motion perception test; and
assessing presence, absence or level of at least one of (i) demyelination and (ii) remyelination, based on said response.

2. The method of claim 1, wherein said determining said subject response comprises applying a scoring procedure to assign a score to said response.

3. The method of claim 1, further comprising correlating said response to visual evoked potentials latency.

4. The method of claim 3, further comprising assessing prolongation of said visual evoked potentials latency.

5. The method of claim 1, being executed while said subject is in an acute phase of optic neuritis.

6. The method of claim 1, wherein said subject has an optic neuritis history, and the method is executed at least one month following an acute phase of said optic neuritis.

7. The method of claim 1, wherein said subject has an optic neuritis history, wherein the method is executed less than two months following an acute phase of said optic neuritis, and wherein the method comprises predicting visual recovery of the subject at a future time.

8. The method of claim 7, wherein said prediction is based on a predetermined recovery rate.

9. The method of claim 1, wherein said motion perception test comprises a motion detection test.

10. The method of claim 9, wherein said motion detection test comprises displaying a stimulus selected from the group consisting of a coherent moving dot array and a collection of stationary dots.

11. The method of claim 10, wherein said motion detection test comprises displaying a plurality of stimuli, each stimulus consisting of a coherent moving dot array characterized by a different moving velocity.

12. The method of claim 1, wherein said motion perception test comprises an object from motion (OFM) extraction test.

13. The method of claim 12, wherein said OFM extraction test comprises displaying at least one OFM stimulus consisting of an array of dots outlining a patterned object and being at a relative motion relative to a patterned background, said patterned object and said patterned background being characterized by the same pattern and being indistinguishable in the absence of said relative motion.

14. The method of claim 13, wherein said at least one OFM stimulus comprises a plurality of OFM stimuli, each being characterized by a different motion velocity.

15. A method of assessing the effect of a treatment, comprising:
administering to a subject a drug identified for the treatment of demyelinating condition;
executing the method of claim 1; and
assessing the effect of said drug based, at least in part, on said presence, absence or level of said demyelination and/or remyelination.

16. A system for diagnosis, comprising a display device and a data processor configured for displaying a motion perception test, receiving a subject response to said motion perception test, determining presence, absence or level of at least one of (i) demyelination and (ii) remyelination, and generating output pertaining to said presence or absence.

17. A computer software product, comprising a computer-readable medium in which program instructions are stored, which instructions, when read by a computer, cause the computer to display a motion perception test, to receive a subject response to said motion perception test, to determine presence, absence or level of at least one of (i) demyelination and (ii) remyelination, and to generate output pertaining to said presence, absence or level.
